(19) United States
(12) Patent Application Publication
(10) Pub. No.: US 2015/0320560 A1
(43) Pub. Date: Nov. 12, 2015

(54) RADIOLUCENT CRANIAL IMPLANTS FOR NEURAL APPLICATIONS

(71) Applicant: Massachusetts Institute of Technology, Cambridge, MA (US)

(72) Inventors: Grant Haverstock Mulliken, Brighton, MA (US); Narcisse Pascal Bichot, Medford, MA (US); Robert Desimone, Brookline, MA (US)

(21) Appl. No.: 14/412,092

(22) PCT Filed: Jul. 3, 2013
(86) PCT No.: PCT/US13/49277

§ 371 Date: Dec. 30, 2014

Related U.S. Application Data
(60) Provisional application No. 61/667,737, filed on Jul. 3, 2012.

(51) Int. Cl. A61F 2/28; A61B 19/00

(52) U.S. Cl. CPC A61F 2/2875; A61B 19/20; A61B 2019/208

(57) ABSTRACT

Cranial implants, kits including cranial implants, and methods of making cranial implants are described. The cranial implants may be radiolucent. The cranial implants may have contact surfaces complementary in shape to measured portions of a surface of a specific cranium. The radiolucent cranial implants may include carbon-PEEK.

### RELATED APPLICATIONS

This application claims priority to U.S. Provisional Application No. 61/667,737 filed Jul. 3, 2012, the contents of which are incorporated herein in their entirety.

### STATEMENT OF GOVERNMENT INTEREST

This invention was made with government support under Grant Nos. F32 EY020692, R01 EY017292 and R01 EY017921 awarded by the National Institutes of Health. The government has certain rights in this invention.

### BACKGROUND

Currently, cranial implants used for neurophysiological investigation are usually made from material such as titanium, polyetherimide and radiolucent ceramic. Although titanium implants are bio-compatible and strong, they are known to create significant artifacts in structural MRI images, significantly hindering the ability of researchers and clinicians to visualize tissue proximal to the implant. Researchers have typically used plastic or ceramic materials for applications involving functional and structural MRI imaging, but these materials are fragile and suboptimal for long-term implantation.

Many cranial implants employ dental acrylic (e.g., methyl methacrylate) to serve as a bonding agent to secure the implant to the skull. For example, when implanting a standard recording chamber without feet/legs, numerous screws are placed into the bone in the area surrounding the chamber, serving as flanking anchors. The chamber is thus indirectly secured to the skull via an acrylic bonding agent that is applied to encase the anchoring screws while adhering to the edges of the implant. Unfortunately, MMA is known to be toxic and does not bond well to bone, resulting in a progressive degradation of underlying bone, which can lead to failure of implants due to loosening of the anchoring screws. The MMA also enables the growth of granulated tissue between the MMA and the bone that is highly prone to infection, which may jeopardize the general health of the implanted animal.

Some existing types of cranial implants (e.g., recording chambers, stimulation chambers, head posts) rely on laterally extending legs or feet to secure the implant to the cranium. An undesirable consequence of this approach is that the wound margin around the chamber often slides down, or recedes, along the tops of the legs because the skin is prevented from attaching to the bone by the legs. The skin recession leads to exposed bone, which degrades over time, and also increases the likelihood of an infection, jeopardizing the integrity and lifetime of the implant. Some implants with legs and an expanded scaffold of methyl methacrylate (MMA) built around the implant to increase structural support may experience both skin recession over the legs and degradation of bone underlying the MMA.

### SUMMARY

Embodiments include radiolucent cranial implants for neurophysiological research, testing, and/or treatment, surgical kits including such implants, and methods of making such implants. In some embodiments, the radiolucent cranial implant includes a conductive radiolucent material. In some embodiments, the radiolucent cranial implant includes carbon-reinforced PolyEtherEtherKetone (carbon-PEEK). In some embodiments, the radiolucent cranial implant includes a conductive radiolucent material. In some embodiments, a base portion of a radiolucent cranial implant has a contact surface complementary in shape to a measured surface of a portion of a cranium. In some embodiments, a shape of the contact surface is formed based on the measured anatomical data of the surface of the portion of the cranium. In some embodiments, the contact surface includes an osteoconductive coating, such as a hydroxyapatite coating. In some embodiments, the cranial access chamber is configured for chronic attachment to a portion of a cranium. In some embodiments, a base portion of the implant has an outer perimeter shaped to form a gapless interface with surrounding tissue. In some embodiments, the outer perimeter is approximately circular, oval, or elliptical in shape. In some embodiments, the outer perimeter forms a convex shape.

An embodiment includes a radiolucent cranial access chamber with an upper portion and a base portion. The base portion includes a sidewall defining a plurality of apertures extending through the base portion. Each aperture is configured to receive a fixation element. An inner surface of the sidewall and the upper portion define an access channel. The upper portion and the base include a radiolucent material.

In some embodiments, the cranial access chamber is configured to receive at least a portion of a multi-channel microdrive for guiding one or more electrodes. In some embodiments, the cranial access chamber is a recording chamber. In some embodiments, the cranial access chamber is a stimulation chamber. In some embodiments, the cranial access chamber is configured to receive one or more guide elements, each guide element defining one or more electrode guide holes.

In some embodiments, the cranial access chamber is configured for electrophysiological recordings. In some embodiments, the chamber is configured for delivery of one or more biologically or pharmacologically active agents to underlying brain tissue. In some embodiments, the chamber is configured for optical stimulation of underlying brain tissue via optical fibers.

In some embodiments, the cranial access chamber also includes a lid configured to cover the upper portion of the access channel. In some embodiments, the cranial access chamber is configured for chronic attachment to a portion of a cranium without the use of an adhesive or bonding agent.

Another embodiment includes a radiolucent head post including an upper portion and a base portion. The base portion defines a plurality of apertures extending through the base portion. Each aperture is configured to receive a radiolucent fixation element. The base portion has a contact surface complementary in shape to a measured surface of a portion of a cranium. At least the base portion of the head post includes a radiolucent material.

An embodiment includes a radiolucent cranial implant kit having a cranial access chamber including a carbon-PEEK material and a head post including a carbon-PEEK material.

An embodiment includes a radiolucent cranial implant kit including a cranial access chamber including a carbon-PEEK material and microdrive body including a carbon-PEEK material.

Any of the radiolucent cranial implant kits may further include a plurality of self-tapping screws including a carbon-PEEK material.

Any of the radiolucent cranial implant kits may also include a multi-channel microdrive configured to be received in the access channel and configured to position one or more electrodes in underlying brain tissue.

Any of the radiolucent cranial implant kits may also include one or more guide elements configured to be received by the access channel, each guide element defining one or more electrode guide holes.

Any of the radiolucent cranial implant kits may further include a drill bit sleeve having an outer diameter complementary to a diameter of an aperture of the cranial access chamber. In some embodiments, a drill bit sleeve has an outer diameter and an inner diameter configured for maintaining an alignment of a drill bit within the sleeve while the drill bit and sleeve combination extend through an aperture of the cranial access chamber or of the head post.

Another embodiment is a method of manufacturing a cranial implant. In some embodiments, the cranial implant is a radiolucent cranial implant. The method includes creating a computer-aided design representation of a cranial implant in which a contact surface of a base portion of the cranial implant is complementary in shape to measured anatomical data regarding a portion of a surface of a cranium. The method also includes forming the cranial implant from a radiolucent material based on the computer-aided design representation.

In some embodiments, the formed cranial implant is configured to form a gapless interface with an underlying surface of the cranium when implanted on the portion of the surface of the cranium.

In some embodiments, forming the cranial implant from a radiolucent material based on the computer-aided design representation includes creating a computer-aided manufacturing file for the cranial implant based on the computer-aided design representation of the cranial implant.

In some embodiments, the method further includes creating a stereolithographic computer-aided design representation of at least a portion of a surface of a cranium based on measured anatomical data. The stereolithographic computer-aided design representation of at least the portion of the surface of the cranium is used to create the computer-aided design representation of the cranial implant. In some embodiments, creating a stereolithographic computer-aided design representation of at least a portion of a surface of a cranium based on measured anatomical data includes segmenting the measured anatomical data to identify the surface of the cranium.

In some embodiments, the method also includes obtaining the measured anatomical data regarding the portion of the surface of the cranium.

In some embodiments, forming the cranial implant from the radiolucent material based on the computer-aided design representation includes machining the cranial implant from a radiolucent material using a multi-axis computer numerical control machine tool.

In some embodiments, the measured anatomical data was obtained using magnetic resonance imaging. In some embodiments, the measured anatomical data was obtained using CT-scanning.

In some embodiments, the cranial implant is formed of a material including carbon-PEEK.

An embodiment is a method of making a radiolucent cranial implant that includes obtaining a radiolucent cranial implant having a contact surface. The method also includes creating a CAD representation of a customized contact surface for the implant having a shape complementary to measured anatomical data regarding a portion of a surface of a specific cranium. The method further includes modifying the contact surface of the obtained cranial implant based on the CAD representation of the customized contact surface.

In some embodiments, the measured anatomical data is MRI data of the specific cranium. In some embodiments, the measured anatomical data is X-ray data of the specific cranium.

In some embodiments, the cranial implant is a cranial access chamber. In some embodiments, the cranial implant is a head post.

### BRIEF DESCRIPTION OF THE DRAWINGS

These figures are intended to illustrate various embodiments and are not intended to depict relative sizes and dimensions, or to limit the scope of examples or embodiments.

FIG. 1 is a perspective view of a radiolucent cranial access chamber, in accordance with an embodiment.

FIG. 2 is a top view of the radiolucent cranial access chamber of FIG. 1.

FIG. 3 is a side cross-sectional view along the 3A-3A plane of the radiolucent cranial access chamber of FIG. 2.

FIG. 4 is a side view of a radiolucent cranial access chamber having a contact surface complementary in shape to a measured portion of a cranium, in accordance with an embodiment.

FIG. 5 is a side view of a radiolucent cranial access chamber having a contact surface complementary in shape to a measured portion of a cranium and having an access port oriented approximately parallel to a normal to the measured surface portion of the cranium.

FIG. 6 is a perspective view of a radiolucent cranial access chamber, in accordance with an embodiment.

FIG. 7 is a perspective view of a radiolucent cranial access chamber mounted on a model of a cranium, in accordance with an embodiment.

FIG. 8 is a perspective view of a radiolucent cranial access chamber having a contact surface complementary in shape to a measured portion of a cranium, in accordance with an embodiment.

FIG. 9 is a bottom perspective view of a radiolucent fixation element, in accordance with an embodiment.

FIG. 10 is a top view of the radiolucent fixation element of FIG. 9.

FIG. 11 is a side view of the radiolucent fixation element of FIG. 9.

FIG. 12 is a cross-sectional view of a prior art access chamber implanted on a cranium.

FIG. 13 is a top view of the prior art access chamber of FIG. 12 implanted on a cranium.

FIG. 14 is a cross-sectional view of the exemplary cranial access chamber of FIGS. 1-3 implanted on a cranium.

FIG. 15 is a perspective view of a microdrive body, in accordance with an embodiment.

FIG. 16 is a top view of the microdrive body of FIG. 15.

FIG. 17 is a side cross-sectional view of the microdrive body of FIG. 15.

FIG. 18 is a top view of a microdrive body for receiving sixteen shuttles, in accordance with an embodiment.

FIG. 19 is a side cross sectional view of an assembly including a radiolucent cranial access chamber, a microdrive body, microdrive shuttles, and a lid, in accordance with an embodiment.

FIG. 20 is a top perspective view of the assembly of FIG. 19.

FIG. 21 is a top view of a ring printed circuit board (PCB) of the recording assembly of FIG. 19.

FIG. 22 is an image of an assembly including a cranial access chamber and microdrive body implanted on a skull model, in accordance with an embodiment.

FIG. 23 is an exploded perspective view of a stimulation assembly including a cranial access chamber for stimulation chamber, electrode guides and a lid, in accordance with an embodiment.

FIG. 24 is a perspective view of the stimulation assembly of FIG. 19 with the stimulation chamber and lid depicted as translucent for illustrative purposes.

FIG. 25 is a schematic side view of a radiolucent head post having laterally extending legs, in accordance with an embodiment.

FIG. 26 is schematic top view of the radiolucent head post of FIG. 25.

FIG. 27 is a schematic perspective view of a radiolucent head post with shaped legs and a shaped contact surface, in accordance with an embodiment.

FIG. 28 is a side view and detail of the radiolucent head post of FIG. 27 positioned on a cranial model.

FIG. 29 is schematic top view of a radiolucent head post that does not have laterally extending legs, in accordance with an embodiment.

FIG. 30 is schematic perspective view of the radiolucent head post of FIG. 29.

FIG. 31 is an image of an example head post positioned on a skull model, in accordance with an embodiment.

FIG. 32 is a flow diagram of a method of making a radiolucent cranial implant in accordance with an embodiment.

FIG. 33 is a flow diagram of a method of modifying a radiolucent cranial implant in accordance with an embodiment.

FIG. 34 is a perspective computer model image of measured anatomical data, a corresponding a head post, and two access chambers, in accordance with an embodiment.

FIG. 35 is a perspective computer model image of smoothed anatomical data, a corresponding a head post, and two access chambers, in accordance with an embodiment.

FIG. 36 is a perspective image of a head post and two access chamber recording chambers produced based on the model of FIG. 36.

FIG. 37 is a perspective image of the brain as targeted by the first access chamber.

FIG. 38 is a perspective image of the brain as targeted by the second access chamber.

FIG. 39 depicts an exemplary computer system suitable for implementation of some embodiments.

FIG. 40 depicts an exemplary network environment suitable for a distributed implementation of some embodiments.

Additional features, functions and benefits of the disclosed methods, systems and media will be apparent from the description which follows, particularly when read in conjunction with the appended figures.

### DETAILED DESCRIPTION

Some embodiments provide cranial implants that are radiolucent (i.e., do not significantly impair visualization of biological structure during MRI, CT and X-ray scans), and made from a biologically compatible material (e.g., carbon-reinforced PolyEtherEtherKetone (carbon-PEEK)) for providing long-term access to brain areas for neurophysiological recording, stimulation and/or drug-delivery.

An example radiolucent cranial access chamber 10 is depicted in FIGS. 1-3. The cranial access chamber 10 includes an upper portion 12 and a base portion 14 including a sidewall 16. In some embodiments, the upper portion 12 and the base portion 14 are one unitary piece, (e.g., are machined as one part from a single block of material). In other embodiments, the upper portion and the base portion are separate pieces joined together or configured to join together.

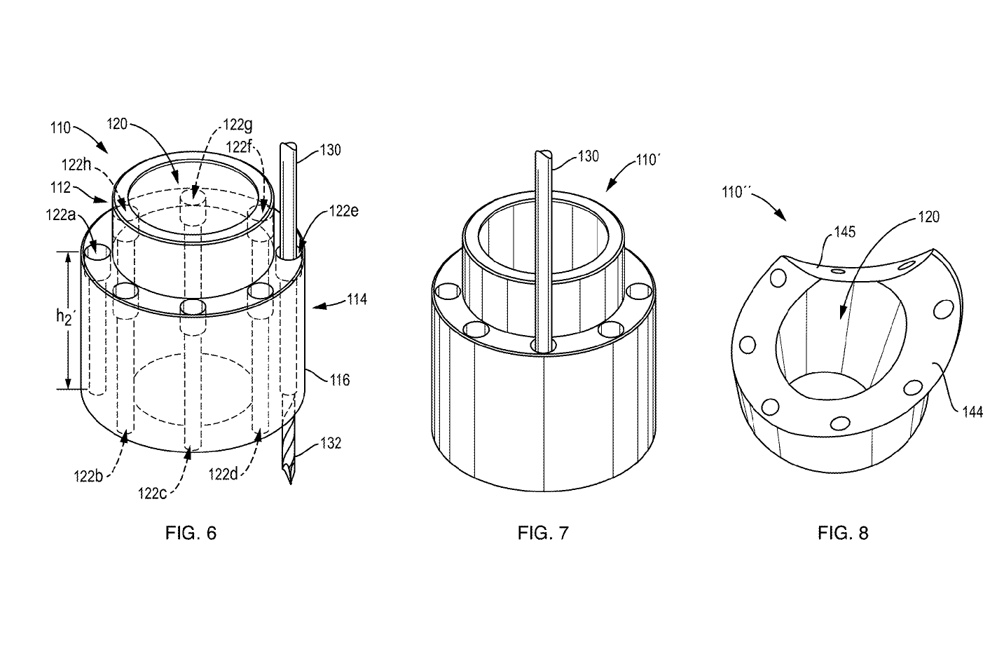

At least the base portion 14 of the cranial access chamber includes a radiolucent material. In some embodiments, both the base portion 14 and the upper portion 12 include a radiolucent material. The upper portion 12 and an inner sidewall surface 18 define an access channel 20. The sidewall 16 of the base portion defines a plurality of apertures 22a, . . . 22h, extending through the base portion 14, each configured to receive a fixation element for affixing the cranial access chamber 10 to a cranium (e.g., see radiolucent self-tapping screw 70 described below with respect to FIGS. 6-8). In cranial access chamber 10, the apertures 22a, . . . 22h, which may be screw guide holes, are embedded within the sidewall 16 of the chamber itself. Although access chamber 10 is depicted with eight apertures, in other embodiments, an access chamber may have more or fewer apertures depending on factors such as the size of the access chamber, the type of fixation elements, the spatial arrangement of apertures, etc. As depicted, in some embodiments, the upper portion may include grooves 26a, 26e for access to corresponding apertures. When the radiolucent cranial access chamber 10 is affixed to a cranium, the access channel 20 provides access to the cranium, or if a portion of the cranium has been removed through a craniotomy, to the underlying brain.

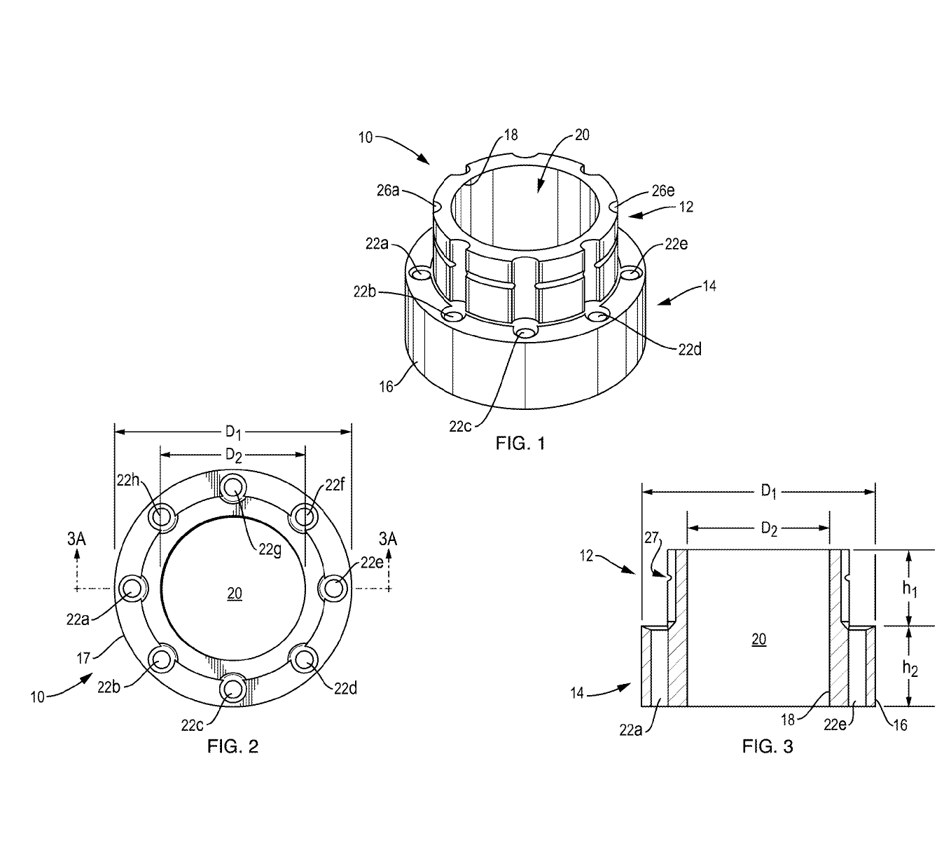

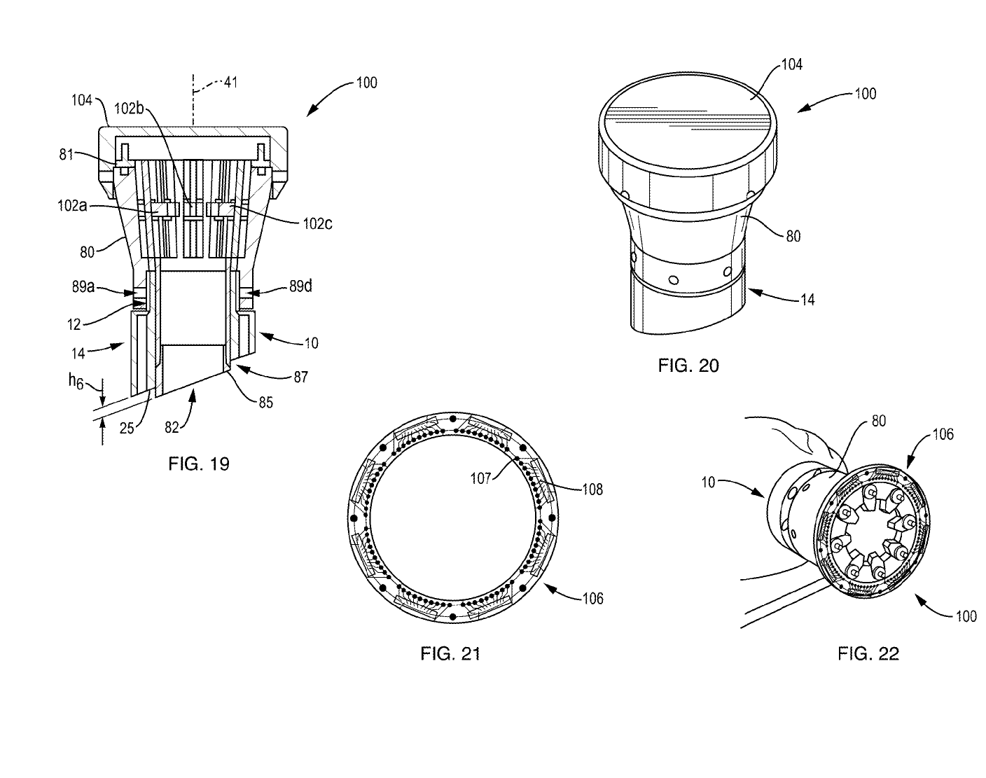

By embedding the screw guide holes in the sidewall 16, the radiolucent access chamber 10 can be secured to the cranium without employing protruding feet or legs. As illustrated by the top view of FIG. 2, an outer perimeter 17 of the base portion has an overall convex shape that promotes a smooth and uninterrupted interface between the chamber 10 and a surrounding wound margin, as compared to prior art configurations with protruding feet/legs. In some embodiments, the outer perimeter is approximately circular, oval or elliptical in shape. In some embodiments, the outer perimeter forms a convex shape other than a circle, oval or ellipse (e.g., stimulation chamber 200 of FIGS. 19-20).

Some embodiments of radiolucent cranial implants described herein (e.g., cranial access chambers for recording or for stimulation, fixation devices, head posts), are formed, at least in part from carbon-PEEK or materials that include carbon-PEEK. Carbon-PEEK is radiolucent, comparable in strength to titanium, less dense than titanium, and is biocompatible. Carbon-PEEK may be particularly suitable for use in cranial implants, (e.g., cranial access chambers for recording and/or stimulation, head posts, skull screw), because the elastic modulus of carbon-PEEK can be made similar to that of bone, reducing the likelihood of stress shielding, which can lead to bone degradation. Further, carbon-PEEK is conductive, meaning that a radiolucent cranial access chamber made from a carbon-PEEK material could function as a Faraday cage for shielding electronics within the access channel. In some embodiments, other radiolucent materials that may be included for cranial implants instead of, or in addition to, carbon-PEEK include, but are not limited to PEEK, polyoxymethylene, polyetherimide and various ceramics.

As depicted in FIGS. 1 and 3, a bottom surface 24 of the cranial access chamber 10 is flat. However, the bottom surface 24 may be formed or modified to be a contact surface complementary in shape to a measured surface of a portion of cranium, which would increase or maximize the contact area between skin and underlying bone in a region proximal to the implant to reduce skin recession and promote a healthier percutaneous margin. Further, the bottom surface 24 is complementary in shape to a measured surface of a portion of a cranium and may enable the implant to be mounted more securely without overhangs or gaps between the implant and the underlying bone.

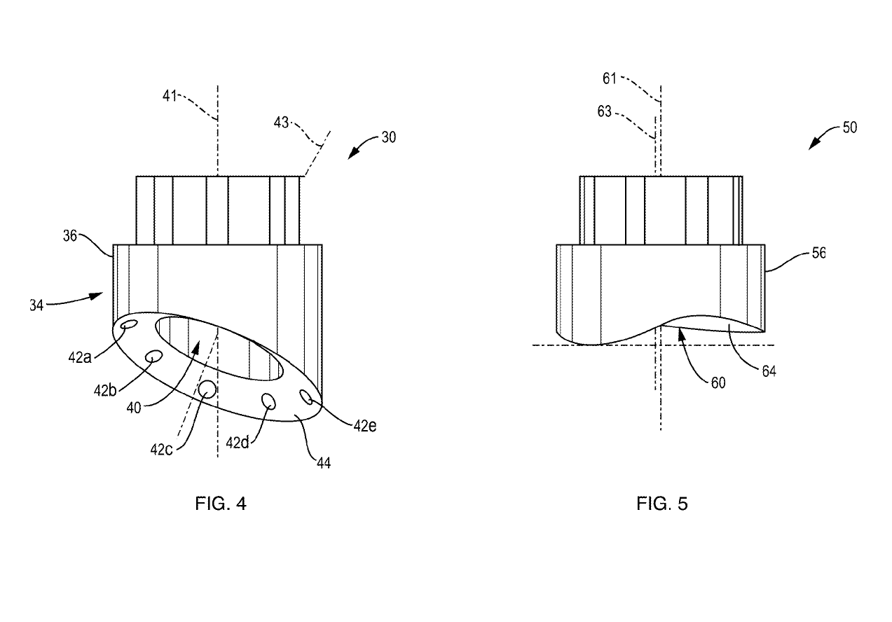

For example, FIG. 4 depicts a cranial access chamber 30 that has a contact surface 44 complementary in shape to a measured surface of a portion of a cranium. The shape of the contact surface 44 corresponds to a portion of cranium through which the lateral intraparietal sulcus (LIP) can be accessed. As depicted in FIG. 4, apertures 42a . . . 42e extend through a base portion 34 of the access chamber 30 and through the contact surface 44. As another example, FIG. 5 depicts a cranial access chamber 50 that has a contact surface 64 complementary in shape to a measured surface of a portion of a cranium through which the extrastriate visual cortical area V4 can be accessed. As depicted in FIG. 4, in some embodiments, a bottom surface 44 of the chamber is shaped such that an axis 41 of an access port 40 of the chamber has an orientation 43 significantly different from the normal 43 of the cranium surface for targeting particular brain structures. As illustrated by FIG. 5, in some embodiments, radiolucent cranial access chamber 60 may have a base 54 with a contact surface 64 shaped such that an axis 61 of an access channel 60 of the chamber may be oriented approximately parallel to a normal 63 of the portion of the surface of the cranium.

In FIGS. 4 and 5, the contact surface 44, 64 having a shape complementary to a measured surface of a portion of a cranium enables the contact surface of the chamber to form a “gapless” interface with the underlying cranial surface. In some embodiments, gaps between the contact surface and the underlying cranial surface are less than 500 p.m. In some embodiments, gaps between the contact surface and the underlying cranial surface are less than 200 μm. In some embodiments, gaps between the contact surface and the underlying cranial surface are less than 50 μm. In some embodiments, the largest gap between the contact surface and the underlying cranial surface falls within a range of 40 to 400 μm. In some embodiments, the largest gap between the contact surface and the underlying cranial surface falls within a range of 40 to 200 μm. The “gapless” interface enables fixation of the access chamber to the cranium without the use of bonding agents such as dental acrylic, which are toxic and promote bone degradation.

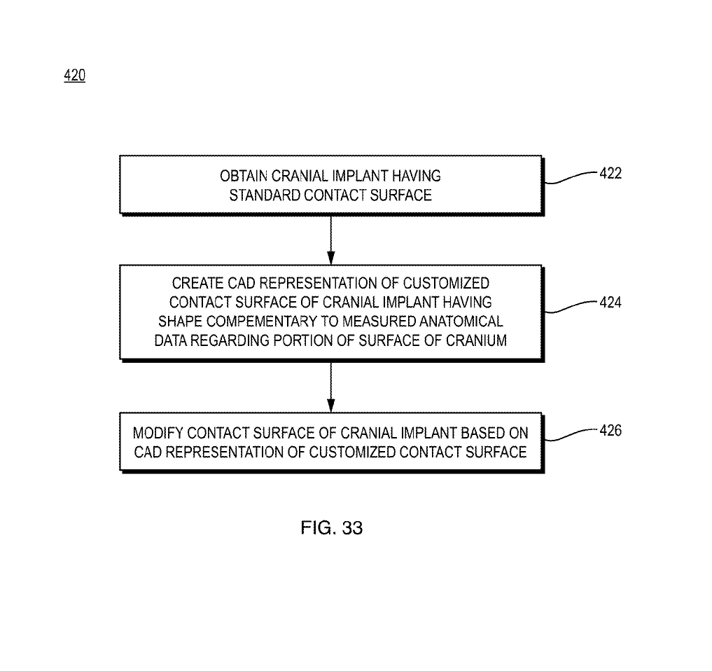

The measured surface of a portion of a cranium may have been measured using any technique that provides sufficiently detailed and precise information regarding the cranium surface to create an implant having a contact surface that can form a “gapless” interface with the cranium (e.g., magnetic resonance imaging (MRI), x-ray, computed tomography (CT) x-ray scanning). Further details regarding a method of making an implant having a contact surface complementary in shape to a measured underlying cranium surface are described below with respect to FIGS. 33-36 and the examples below.

In some embodiments, the contact surface of the chamber includes a coating. In some embodiments, other surfaces of a radiolucent chamber may also have a coating. In some embodiments, the coating is an osteoconductive coating. For example, a coating including hydroxyapatite may improve the osseointegrative properties of the contact surface.

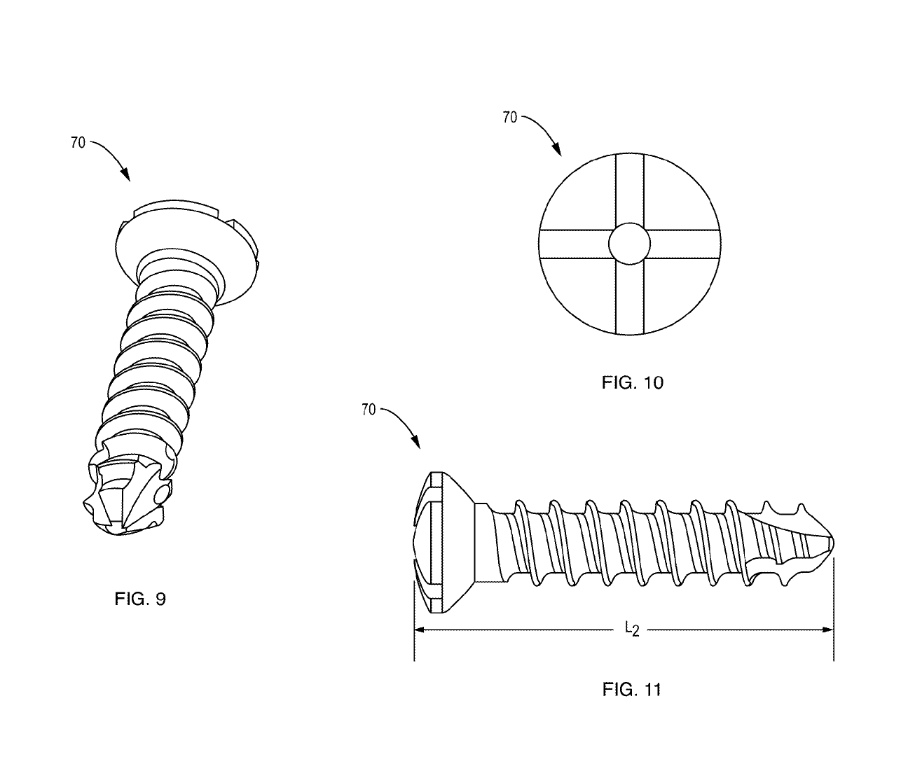

FIG. 6 schematically depicts another embodiment of a radiolucent cranial access chamber 110 that includes an upper portion 112 and a base portion 114 including a sidewall 116. At least the base portion 114 of the cranial access chamber includes a radiolucent material. The upper portion 112 and an inner sidewall surface 118 define an access channel 120. The sidewall 116 of the base portion defines a plurality of apertures 122a, . . . 122h, extending through the base portion 114, each configured to receive a fixation element for affixing the cranial access chamber 110 to a cranium (e.g., see radiolucent self-tapping screw 70 described below with respect to FIGS. 8-10). In cranial access chamber 110, the apertures 122a . . . 122h, which may be screw guide holes, are embedded within the sidewall 116 of the chamber itself. As compared with chamber 10 of FIGS. 1-3, the apertures 122a . . . 122h of chamber 110 are spaced further from the inner sidewall surface 118 that defines the access channel 120, which corresponds to the location of the craniotomy during use. This results in the fixation elements being anchored in bone further from the craniotomy, which may be stronger than bone located closer to the craniotomy.

Chamber 110 also has a lower portion 114 with a height h_{2′} greater than the height h₂ of the lower portion 14 of chamber 10 of FIGS. 1-3. Aperatures 122a . . . 122h can be used to guide a drill bit for drilling holes in a cranium for fixation elements. The increased length of the apertures 122a . . . 122h due to the increased length of the lower portion 114 may provide greater stability for guiding a drill bit. In some embodiments, a drill bit sleeve 130 (also known as a drill bit collar) with an outer diameter corresponding to an inner diameter of the aperture may be employed to guide a drill bit 132.

FIG. 6 depicts the chamber before it is modified to have a contact surface complementary to a measured surface of a cranium. FIG. 7 is an image of a radiolucent cranial access chamber 110′ produced based on the model of chamber 110 of FIG. 6, but modified to have a contact surface complementary to a measured surface of a cranium. The chamber 110′ is positioned on a wax model of the cranium with the drill bit sleeve 130 positioned in one of the apertures.

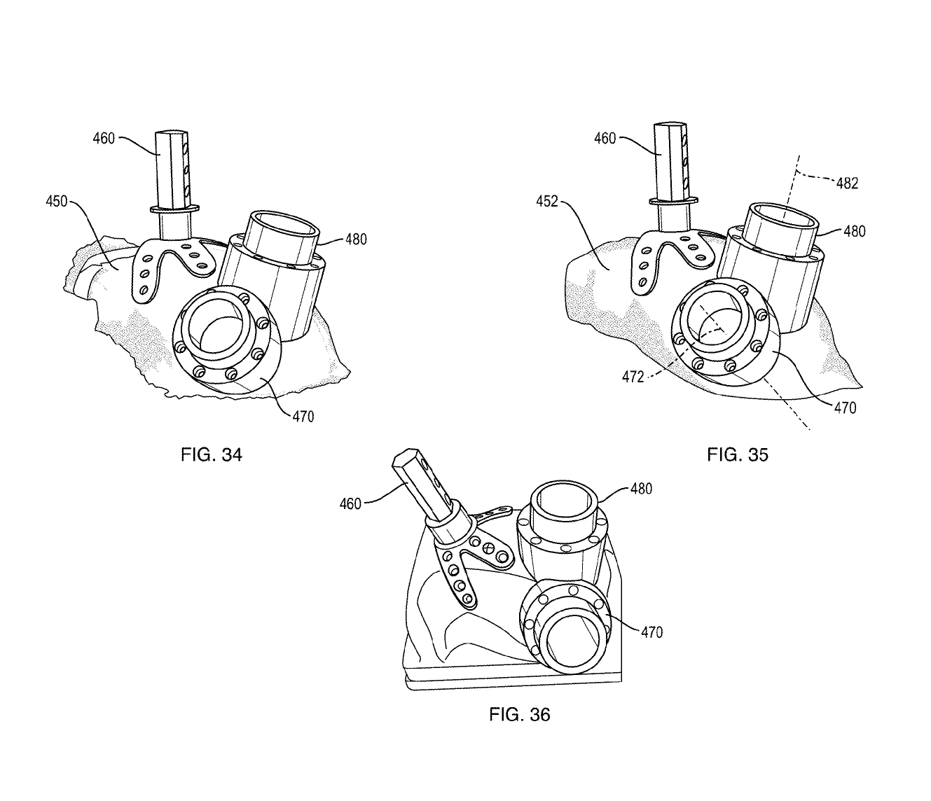

FIG. 8 is an image of another radiolucent cranial access chamber 110″ similar to chamber 110 of FIG. 6, but modified to have a customized contact surface. Specifically, chamber 110″ includes a contact surface 144 having a shape complementary to a measured surface of a portion of a cranium that enables the contact surface of the chamber 110″ to form a “gapless” interface with the underlying cranial surface. In some embodiments, a portion 145 of the contact surface 144 be modified to accommodate an adjacent implant (see FIGS. 34-38 below depicting adjacent chambers).

FIGS. 9-11 illustrate a radiolucent fixation element (e.g., bone screw 70) for affixing a cranial access chamber, head post or other cranial implant to bone. The radiolucent fixation element is itself a cranial implant. In some embodiments, the radiolucent fixation element 70 is carbon-PEEK fixation element (e.g., a carbon-PEEK skull screw). In some embodiments, the bone screw 70 is configured to be self-tapping (i.e., creates its own thread as it is advanced into bone) and therefore does not require that a surgeon tap the drilled screw hole prior to insertion. In some embodiments, most or all of a radiolucent fixation element 70 may have an osteoconductive coating (e.g., a hydroxyapatite coating).

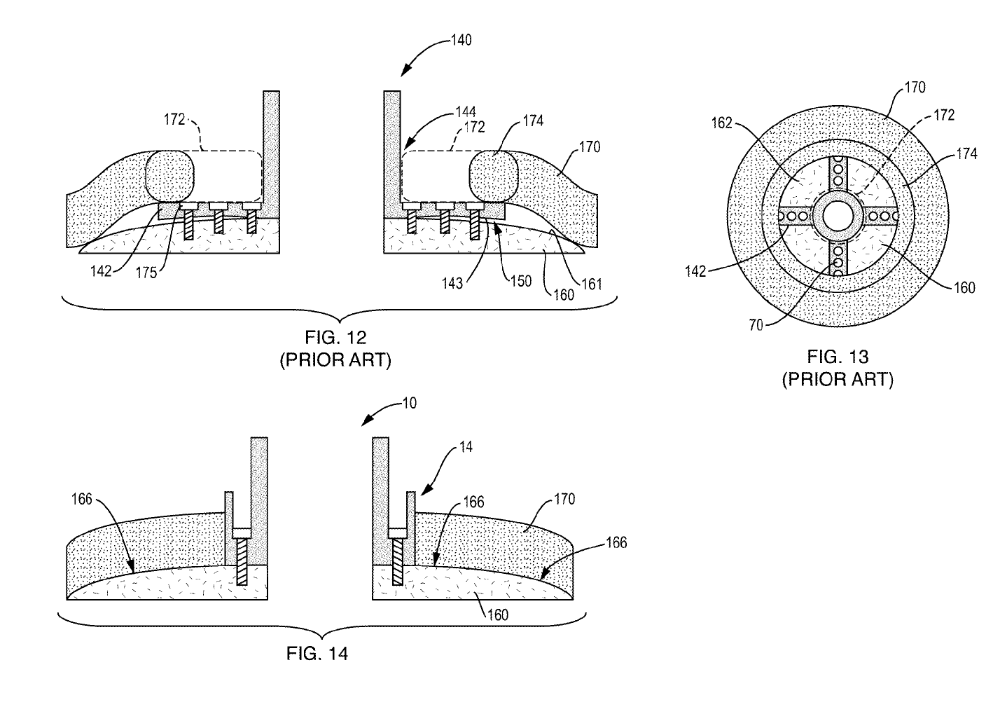

FIGS. 12 and 13 schematically depict a cross-sectional view and a top view, respectively, of a conventional cranial access chamber 140 having legs 142 secured to a cranium 60 by screws 175. In FIGS. 12-14, relative thicknesses and dimensions of various elements are exaggerated for illustrative purposes. Initially, after implantation, the skin 170 overlies the legs 150 and screws 70 and is tightly closed around a base 144 of the chamber as illustrated by the broken line 172 in FIGS. 9 and 10. As depicted in FIG. 12, the legs 142 separate the skin 170 from the underlying cranium 160. Over time (e.g., months to years), the skin 170 recedes exposing part or all of the legs 142 and portions 162 of the cranium. For example, recession of 5-10 mm on average within the course of a year is common. Further, more extreme skin retraction can occur over longer periods of time (e.g., receding 2.8-3.2 cm radially, which may fully expose the legs). The receding skin is believed to be caused by an inability of the skin to bond naturally to the underlying bone due to the raised surface of the leg and due to excessive tension/pressure on the overlying skin. In some conventional implants, a leg is 1.5 mm thick and 6.2 mm wide. The forces produced by the tension in the overlying skin can even result in the leg protruding directly through the skin, independent of whether or not the skin is receding.

As schematically illustrated in FIGS. 12 and 13, unhealthy wound margins 174 commonly develop around the periphery of implants 140 having protruding legs. As the skin 170 radially recedes along the implant legs 142, granulated tissue forms at the skin wound margin 174 around the implant (e.g., chamber 140) due to tissue movement increasing tension in skin overlying the legs. The recession and resulting exposed bone 162, and unhealthy wound margins 174 can lead to frequent infections.

Also, as depicted in FIG. 12, significant gaps 150 commonly occur between a bottom surface 143 of legs and a cranial surface 161 with conventional implants (e.g., gaps are commonly 1-2 mm). A bone void filler may be applied to fill the gaps. In situations where MMA is also applied around the implant, problems with skin recession and infections can become exacerbated. Over time, the wound margin around the edge of the MMA boundary will generally become excessively large, (e.g., 2-6 mm of granulated tissue), which may be continuously infected and prone to hemorrhaging. Furthermore, granulated tissue often grows beneath the MMA surface, forming a channel/path for infection beneath the surface of the implant, limiting the lifetime of the implant substantially. The ingrowth of tissue between MMA and the cranium can also act a wedge that compromises the securing strength of the MMA implant to the cranium.

FIG. 14 schematically depicts a simplified cross-sectional view of the cranial access chamber 10 affixed to a cranium 160 and surrounding skin, in accordance with an embodiment. In contrast to the conventional chamber 140 with legs 142, which is depicted in FIGS. 12 and 13, chamber 10 provides an uninterrupted skin-bone interface 166 right up to the edge of the base 14 of the chamber itself, allowing for the skin 170 to attach/bind naturally to the underlying bone 160 around the chamber perimeter to promote healing. Studies using an example chamber that provided an uninterrupted skin-bone interface resulted in little to no skin recession over a period of months to years as described below in the Examples section.

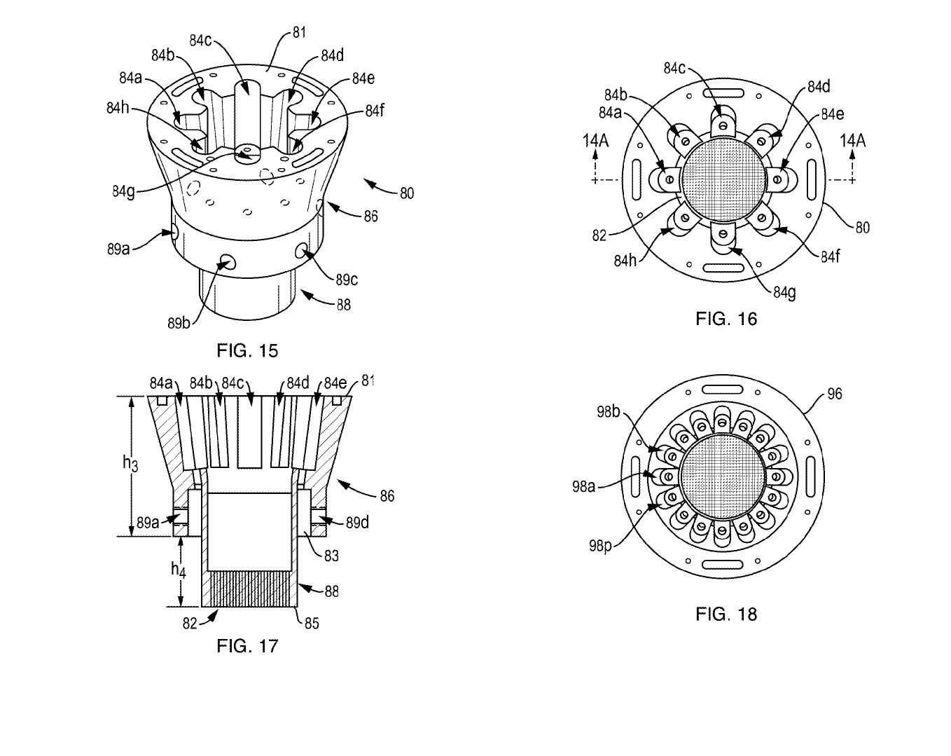

In some embodiments, the radiolucent cranial access chamber 10 is configured to receive at least a portion of a multi-channel microdrive for guiding one or more electrodes for recording electrical signals. FIGS. 15-17 depict a microdrive body 80 that can be received by the radiolucent cranial access chambers of FIGS. 1-5. Such a multi-channel microdrive may be employed in a chamber used for recording electrical signals (e.g., a recording chamber), in a chamber used for electrical stimulation (e.g., a stimulation chamber), or in a chamber used for both recording electrical signals and for electrical stimulation.

In some embodiments the microdrive body 80 defines an array of guide holes 82 that provides support channels for guiding microelectrodes into underlying brain tissue. Diameters and spacings of guide holes may vary in different embodiments. In some embodiments, an array of guide holes may include guide holes of multiple different diameters and/or multiple different spacings. As depicted, the array of guide holes 82 is disposed in a proximal portion 88 of the microdrive body. In other embodiments, guide holes may be disposed in other portions of the microdrive body or in multiple different portions of the microdrive body.

As depicted in FIGS. 15-17, the microdrive body 80 also defines receptacles 84a, . . . 84h for receiving microdrive shuttles (see FIG. 18) that advance and retract microelectrodes with respect to the brain tissue. In some embodiments, the distal portion 86 of the microdrive body defines the receptacles 84a, . . . 84h. In FIGS. 15-17, each receptacle 84a, . . . 84h is configured to receive one shuttle. In other embodiments, each receptacle may be configured to receive more than one shuttle, or each shuttle may be configured to be received by multiple receptacles. In the embodiment of FIGS. 15-17, the microdrive body 80 includes eight receptacles for eight shuttles. In other embodiments, a microdrive body may be configured to receive more than eight shuttles or less than eight shuttles. For example, FIG. 18 depicts a microdrive a body 96 that defines receptacles 98a, 98b, . . . 98p for sixteen shuttles.

In some embodiments, a distal portion 86 of the microdrive body defines a recess 83 configured to receive the upper portion 12 of the cranial access chamber as depicted in FIGS. 14 and 16. The distal portion 86 of the microdrive body may define apertures 89a . . . 89d extending to the recess 83. The upper portion 12 of the cranial access chamber may include a groove 27 that aligns with the apertures 89a . . . 89d of the microdrive body when the microdrive body 80 is received by the cranial access chamber 10 (see FIGS. 1, 19). A fixation element, such as a screw or a pin, may be inserted through the apertures 89a . . . 89d to contact the groove 27.

FIGS. 19 and 20 depict an assembly 100 including access chamber 10 and microdrive body 80 with microdrive shuttles 102a, 102b, . . . 102e. The assembly may be a recording assembly (e.g., for recording electrical signals and/or for recording chemical signals), a stimulation assembly (e.g., for electrical stimulation and/or for chemical stimulation), or both. In some embodiments, microelectrodes (not depicted) are back-loaded through the desired grid holes 82 into the interior of the microdrive body 80, and then secured to the shuttles 102a, . . . 102e. Flexible insulated tubing, (e.g., polyimide tubing) may be used to provide a snug fit between the microelectrodes (not depicted) and the corresponding guide holes 82, while providing enough space for the microelectrodes to slide freely inside of the tubing. In some embodiments, each shuttle 102a, 102b, . . . 102e is advanced downward toward brain tissue or retracted upward away from the brain tissue using a screwdriver that rotates the shuttle assembly on a threaded rod. In the embodiment of FIG. 19, each shuttle holds up to four microelectrodes. However, the number of shuttles and the number of microelectrodes per shuttle may be more or fewer. In some embodiments, a distal end of each microelectrode is wired to a VIA hole 107 on an annulus-shaped printed circuit board (PCB) 106 that is mounted to a distal end 81 of the microdrive body 80, (see FIGS. 19, 21). For example, in some embodiments, one end of a 30-gauge copper wire may be soldered to the PCB pad (not depicted) and the other end soldered to a female copper receptacle (not depicted) using a solder sleeve (not depicted), which is then crimped onto the back end of the electrode and secured further using silver paste (not depicted). The PCB layout routes the signal at the VIAs to pins of surface-mounted connectors 108 (e.g., Omnetics connectors) for connection with a neurophysiological data acquisition system, which may interface with front-end pre-amplification and filtering hardware. FIG. 22 is an image of an example assembly 100 including a cranial access chamber 10 and a microdrive 80 with a PCB 106 mounted on a model of a cranium. One of ordinary skill in the art, in view of this disclosure will understand that various different types of shuttles, electrodes and connectors may be employed in various embodiments.

Turning again to FIGS. 19 and 20, the assembly 100 may also include a lid 104 covering a distal end 81 of the microdrive body. In some embodiments, assembly 100 is a chronic recording assembly used for chronic (weeks, months, or years) or acute (daily) neurophysiological recordings.

As also depicted in FIGS. 19 and 20, a proximal end 87 of the microdrive body may have a surface 85 that is not normal with respect to the axis 41 of the access channel of the cranial access chamber. The proximal end 87 of the microdrive body may extend past a contact surface 25 of the base portion 14 of the access chamber by an amount h₆, as depicted in FIG. 19. In some embodiments, a shape of a proximal end surface 85 of the microdrive body corresponds to an underlying anatomical structure (e.g., corresponds to an outer surface of the brain). For example, in some embodiments, a shape of the proximal end surface 85 may enable positioning the surface in physical contact with the underlying brain surface without compressing the underlying brain surface. As another example, a shape of the proximal end surface 85 may enable positioning the surface in physical contact with the underlying brain with slight pressure applied against the brain surface. In some embodiments, a shape of the proximal end surface 85 may enable positioning the surface at a set distance from the underlying brain surface (e.g., less than 2 mm from the underlying brain surface).

The microdrive body may be formed in part or in full from carbon-PEEK, or from a material including carbon-PEEK. Because carbon-PEEK is conductive, a microdrive body formed of carbon-PEEK would serve as a faraday cage to reduce to electrical noise along the signal pathway of the electrodes due to electromagnetic interference, which is commonly encountered in neurophysiological applications, especially those involving recording of microvolt neurophysiological signal. In particular, the most vulnerable part of the signal pathway is often a segment prior to amplification (i.e., the electrode themselves, the PCB and the connecting wires), of which the electrodes and the PCB would be shielded from electromagnetic fields by the carbon-PEEK microdrive body and an associated lid or cover.

In some embodiments, the lid or cover is formed of a conductive material that substantially or completely encloses the top of the assembly and that is in electrical contact with the drive for shielding. In some embodiments, the distal portion of the assembly is substantially or completely enclosed by conductors, other than apertures for contacts, leads, connectors or cables, to act as a Faraday cage.

The conductivity of the carbon-PEEK may vary depending on the carbon content of the carbon-PEEK. For example, unfilled PEEK having no carbon may have relatively low conductivity (e.g., a resistivity on the order of 10¹⁵ to 10¹⁶ Ohm-cm). A carbon-PEEK material with 30% carbon (a 30% filled carbon-PEEK), which has a modulus of elasticity similar to that of bone, would have a higher conductivity (e.g., a resistivity on the order of 10³ to 10⁵ Ohm-cm). A carbon-PEEK material with more than 30% carbon (greater than 30% filled carbon-PEEK) may have an even higher conductivity (e.g., a resistivity on the order of 0.1 Ohm-cm). For example, a 30% carbon filled-PEEK nylon material has been used for an enclosure that achieves 60 dB EMI/RF rejection. Due to the increased conductivity, it is expected that a carbon-PEEK material with a greater carbon fill would achieve better rejection/isolation.

The 30% carbon filled-PEEK composition is useful for anchoring elements (e.g., fixation elements such as screws) and elements under significant mechanical load (e.g., head posts) because this composition matches the modulus of elasticity of bone, reducing the effect of stress-shielding. However, for elements under less mechanical load (e.g., access chambers) and/or elements not in contact with bone (microdrives), matching the modulus of elasticity may be less important. For these elements, a material with greater than 30% carbon fill may be employed for improved electrical isolation despite the mismatch with the modulus of elasticity of the underlying bone.

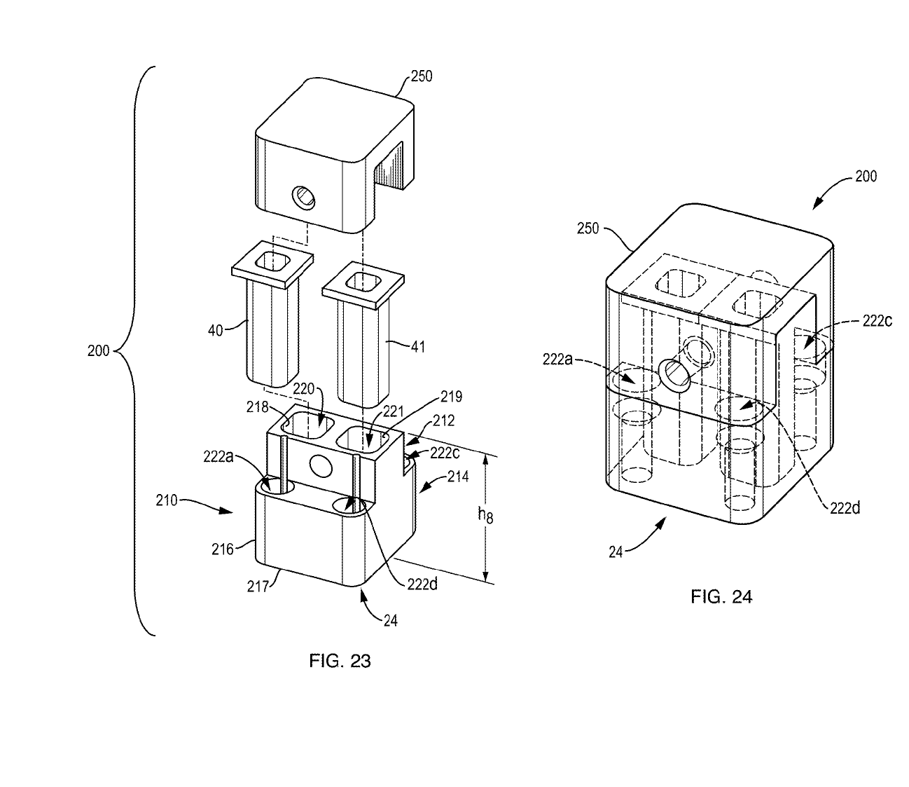

FIGS. 23 and 24 depict a stimulation assembly 200 including another embodiment of a cranial access chamber used for stimulation. Stimulation chamber 210 includes an upper portion 212 and a base portion 214 having a sidewall 216. The sidewall 216 of the base portion defines a plurality of apertures 222a, . . . 222d extending through the base portion 214, each configured to receive a fixation element (e.g., see radiolucent self-tapping screw of FIGS. 6-8) for affixing the stimulation chamber 210 to a cranium. In some embodiments, the upper portion 212 and the base portion 214 may be one unitary piece, (e.g., are machined as one part from a single block of material). In other embodiments, the upper portion and the base portion may be separate pieces joined together and/or configured to join together.

Although stimulation chamber 210 includes four apertures 222a . . . 222d for receiving fixation elements, in other embodiments, the base portion may define more or fewer apertures for receiving fixation elements. In stimulation chamber 210, the apertures 222a, . . . 222d, which may be screw guide holes, are embedded within the sidewall 216 of the chamber itself allowing for a smooth and uninterrupted interface between the edge of the chamber and the wound margin during use. As illustrated by FIGS. 19 and 20, the base portion 214 of the stimulation chamber may have a perimeter 217 in the shape of a rectangle with rounded corners to promote a smooth interface with the surrounding skin. In other embodiments, the perimeter of the base portion could be circular or have another convex shape. As illustrated by FIG. 24, the apertures 222a . . . 222d for receiving fixation elements may be countersunk.

The upper portion 212 and a first inner surface 218 of the sidewall define a first access channel 220. The upper portion 212 and a second inner surface 219 of the sidewall define a second access channel 221. When the stimulation chamber 210 is affixed to a cranium, the first access channel 220 and second access channel 221 provide access to the cranium, or if a portion of the cranium has been removed through a craniotomy, to the underlying brain tissue. Although stimulation chamber 210 has two access channels, in other embodiments, a cranial access chamber used for stimulation may have more or fewer channels.

The first access channel 220 and the second access channel 221 are each configured to receive an electrode guide. The stimulation assembly 200 may include a first electrode guide 240 and a second electrode guide 241 each defining multiple guide channels (not depicted) to align stimulating electrodes (not depicted) with underlying brain structures. For example, the stimulation assembly may be used for bilateral stimulation of reward-related structures of the brain (e.g., nucleus accumbens, ventral tegmental area) near the midline. In some embodiments, the stimulation assembly 200 further includes a lid 250

In some embodiments, at least the base portion 214 of the stimulation chamber 210 is radiolucent. In some embodiments, both the base portion 214 and the upper portion 212 of the stimulation chamber are radiolucent. In some embodiments, the first electrode guide 240 and the second electrode guide 241 also include a radiolucent material. In some embodiments, the lid 250 also includes a radiolucent material.

Although a bottom surface 224 of the base portion 214 in FIGS. 23 and 24 is depicted as flat, in some embodiments, the bottom surface 224 may be formed or modified to be a contact surface complementary in shape to a measured surface of a portion of cranium, which would increase or maximize the contact area between skin and underlying bone in a region proximal to the implant to reduce skin recession and promote a healthier percutaneous margin. In some embodiments, the bottom surface 224 is machined, molded or formed in another manner to match a measured surface portion of the cranium.

Embodiments are not limited to access chambers for stimulating and/or recording electrodes inserted into the brain (i.e., impaling microelectrodes). For example, in some embodiments, one or more stimulating and/or recording electrodes may be positioned on a surface of the brain (e.g., electrocorticography (ECoG) electrodes).

Embodiments are not limited to radiolucent access chambers for electrodes. For example, in some embodiments, the access chamber may be used for stimulation or illumination of portions of the brain using laser light. In an embodiment, an access chamber may house an array of optical fibers for stimulating underlying neural targets of the brain using laser light (e.g., for optogenetics). As another example, the access chamber may house microfluidic channels (e.g., for delivery of a biologically or pharmacologically active agent such as a drug, for chemical stimulation, or for recording chemical signals). In some embodiments, an access chamber may be configured for any combination of impaling electrodes, surface electrodes, optical fibers, and/or fluidic microchannels.

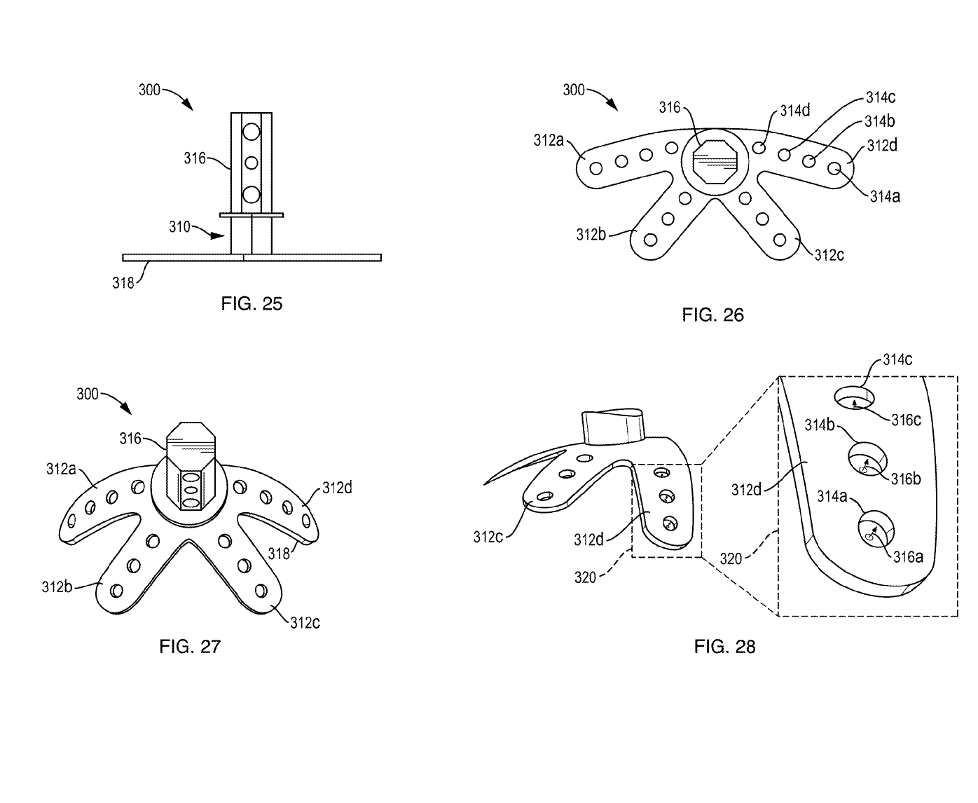

FIGS. 25-28 depict a radiolucent head post 300 for securing a position of a cranium during stimulation, recording or testing (e.g., during measurement of behavioral variable such as head and eye position), in accordance with some embodiments. The head post 300 includes a base portion 310 and one or more legs 312a, 312b, 312c, 312d extending from the base portion. Each leg defines multiple apertures, for example, apertures 314a, 314b, 314c, 314d for receiving fixation elements to secure the head post 300 to the cranium. The base portion 310 and the legs 312a, 312b, 312c, 312d have a contact surface 318 for contacting the underlying cranium. In some embodiments, part or all of the surfaces of the implant (e.g., contact surface 318) include an osteoconductive coating (e.g., a hydroxyapatite coating) to enhance integration of the implant into bone. Such a coating may be more important for load bearing implants, such as head posts, than for non-load bearing implants, such as access chambers.

FIGS. 25 and 26 depict a head post 300 with planar legs 312a-312d and a planar contact surface 318. In some embodiments, the contact surface 318 may be complementary in shape to a measured surface of a portion of a cranium. For example, the legs 312a-312d and the contact surface 318 may be formed or machined based on measured anatomical data, such as MRI or CT data. FIGS. 27 and 28 illustrate a head post with legs 312a-312d and a contact surface 318 shaped to be complementary to a measured surface of a cranium. FIG. 28 schematically illustrates the head post 300 mounted on a model of a cranium. In some embodiments, each aperture (e.g., 314a-314d) is shaped such that the axis 316a-316c of each aperture 316a-316c is perpendicular to the underlying cranial surface, as depicted in detail 320 of FIG. 28. This enables each fixation element to be inserted perpendicular to the underlying cranial surface for more secure mounting.

The radiolucent head post 300 also includes an upper portion 316 configured to couple to a device or system for positioning or maintaining a position of the cranium. Although a head post with lateral extensions attaching to the cranium (e.g., legs or feet) may experience recession of skin overlying the lateral extensions, in implants subject to substantial lateral forces and twisting forces (such as head posts) fixation devices may need to extend laterally over a large area of the cranium to spread the area over which the applied force is exerted.

In some embodiments, at least the base portion 310 and the legs 312a, 312b, 312c, 312d of the head post 300 may be radiolucent. In some embodiments, at least the base portion 310, the legs 312a, 312b, 312c, 312d and a part of the upper portion 316 may be radiolucent. In some embodiments, all of the head post 300 may be radiolucent. In some embodiments, the head post 300 may be formed, at least in part, of a radiolucent material including carbon-PEEK. In some embodiments, the head post 300 may be formed, at least in part, of a conductive radiolucent material. In some embodiments, at least the base portion 310 and the legs 312a, . . . 312d may have a contact surface coated with material that promotes osseointegration and may increase fixation strength.

As noted above, carbon-PEEK has excellent mechanical strength and wear performance, similar to that of titanium, but is less dense than titanium. Carbon-PEEK (e.g., 30% carbon filled PEEK) has a modulus of elasticity closer to that of bone, as compared to the modulus of elasticity of titanium, which may reduce stress shielding. As also noted above, carbon-PEEK is radiolucent to X-ray, CT and MRI scans, which will allow both scientific investigators and clinicians to view underlying and surrounding tissue without occlusion or obstruction from the head post while performing structural and functional imaging.

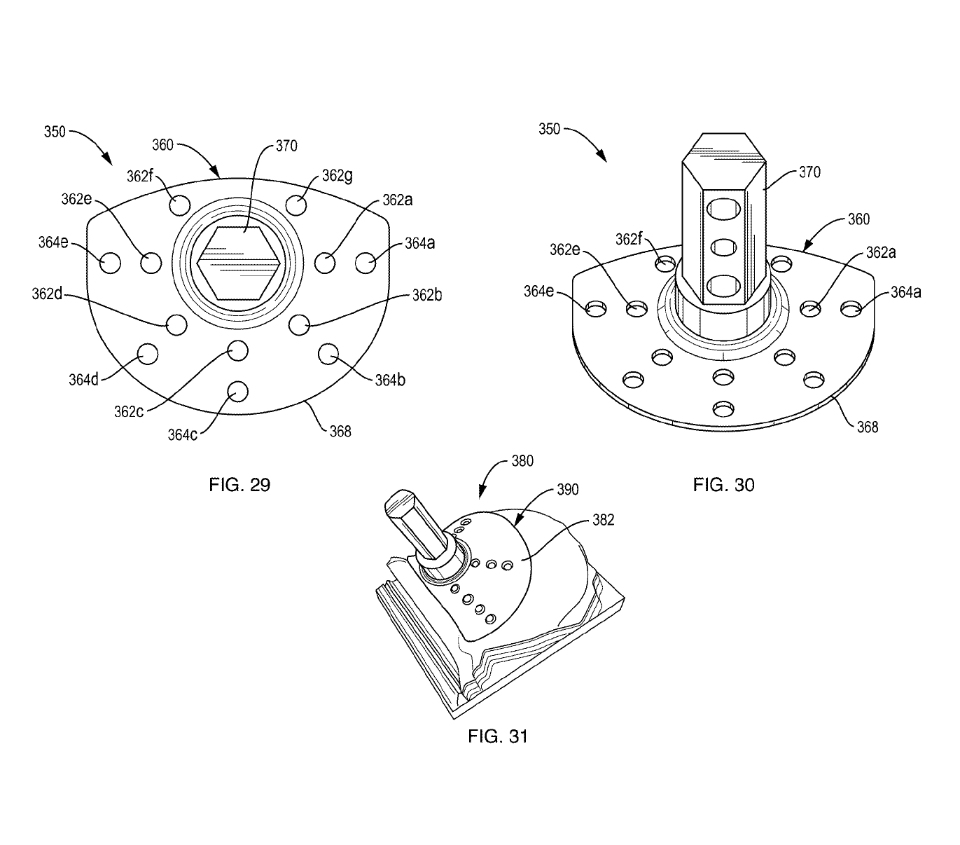

FIGS. 29 and 30 depict another exemplary embodiment of a head post 350 with an upper portion 370 and a base portion 360 that does not include lateral extensions (e.g., legs or feet) for attaching the head post 350 to the cranium. The base portion 360 defines a first set of apertures 362a, . . . 362g, and a second set of apertures 364a, . . . 364e for receiving fixation elements (e.g., self-tapping bone screws). Although head post 350 is depicted with two sets of apertures, in other embodiments, a head post may have more or fewer sets of apertures and may have more or fewer apertures in each set depending on factors such as the size of the head post, the expected forces on the head post, the type of fixation elements, the spatial arrangement of apertures, etc. A perimeter 368 of the base portion is convex to promote a smooth interface with surrounding skin. In some embodiments, a contact surface 366 of the base portion is shaped, formed or modified to be complementary in shape to a measured surface of a portion of cranium, which would increase or maximize the contact area between skin and underlying bone in a region proximal to the heat post 350 to reduce skin recession and promote a healthier percutaneous margin.

FIG. 31 is an image of an example head post 380 positioned on a skull model, in accordance with an embodiment. Head post 380 has a base portion 390 complementary in shape to a measured surface of a portion of a cranium. Base portion 390 of head post 380 covers a larger area that base portion 360 of head post 350 depicted in FIGS. 29-30. Base portion 390 of head post 380 also has a different number and arrangement of apertures 382 as compared with those of head post 350.

Some embodiments include a radiolucent cranial implant kit. The kit may include one or more radiolucent (e.g., carbon-PEEK) cranial access chambers (e.g., cranial access chamber 10, stimulation chamber 210), and a radiolucent (e.g., carbon-PEEK) head post (e.g., head post 300, head post 350). In some embodiments, the radiolucent implant kit includes one or more radiolucent (e.g., carbon-PEEK) cranial access chambers (e.g., cranial access chamber 10, stimulation chamber 210) and one or more microdrive bodies (e.g., microdrive body 80). In some embodiments, the implant kit includes one or more radiolucent cranial access chambers, one or more microdrive assemblies, and a head post. In some embodiments, the cranial implant kit includes a plurality of self-tapping carbon-PEEK screws (e.g., screw 70). In some embodiments the cranial implant kit includes a drill bit sleeve (e.g., drill bit sleeve 130) with an outer diameter selected to correspond to (e.g., be slightly smaller than) a diameter of an aperture of an implant. In some embodiments, the cranial implant kit includes a radiolucent implant (e.g., a carbon-PEEK access chamber or a carbon-PEEK head post), radiolucent fixation elements (e.g., carbon-PEEK screw) and a drill bit sleeve selected to correspond to (e.g., be slightly smaller than) a diameter of an aperture of an implant.

In some embodiments, a contact surface of the cranial access chamber (e.g., contact surface 44 of FIG. 4, contact surface 24 of FIG. 23) and a contact surface of the head post (e.g., contact surface 318 of FIG. 25) include an osteoconductive coating (e.g., a hydroxyapatite coating). In some embodiments, the radiolucent cranial implant kit further includes a multi-channel microdrive configured to be received in an access channel of the cranial access chamber and configured to position one or more electrodes in underlying brain tissue (e.g., microdrive body 80).

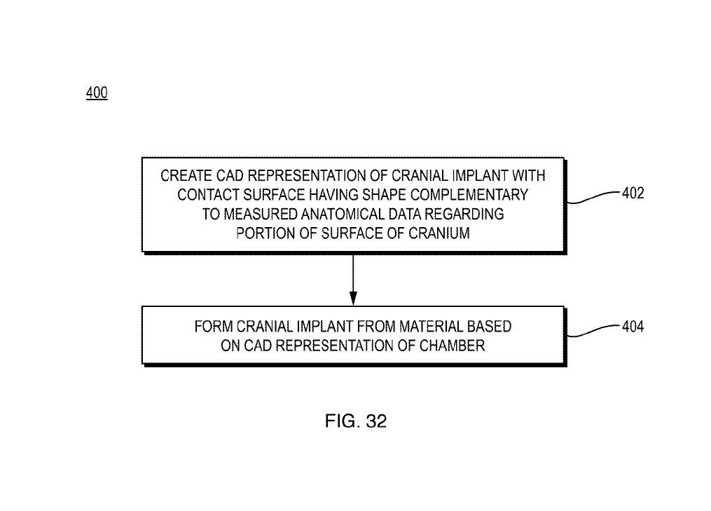

Some embodiments include a method of manufacturing a cranial implant using anatomical data regarding a portion of a surface of a cranium. For example, in method 400 of FIG. 32, a computer-aided design (CAD) representation of cranial implant (e.g., a cranial access chamber or a head post) is created (step 402). For illustrative purposes, the method 400 is described with respect to the reference numbers for cranial access chamber 10 of FIGS. 1-3; however, method 400 may be employed to make other types of cranial access chambers (e.g., a stimulation chamber) or other types of cranial implants (e.g., a head post). In the CAD representation, a contact surface 24 of a base portion 14 of cranial implant 10 is complementary in shape to measured anatomical data regarding a portion of a surface of a cranium. A cranial implant 10 is formed from a material based on the computer-aided design representation (step 404). In some embodiments, the material is a radiolucent material and the implant is a radiolucent implant. In some embodiments, the radiolucent material includes carbon-PEEK. In some embodiments, the implant 10 is configured to form a gapless interface with surrounding tissue when implanted on the portion of the surface of the cranium. For example, the base portion of the implant may have a convex perimeter 17 that does not include lateral protrusions such as legs or feet (see e.g., FIG. 2).

In some embodiments, the method includes creating a computer-aided design representation of the portion of the surface of the cranium based on the measured anatomical data. The computer-aided design representation of at least the portion of the surface of the cranium is used to create the computer-aided design representation of the cranial implant.

In some embodiments, the measured anatomical data was obtained using magnetic resonance imaging. In some embodiments, the measured anatomical data could have been obtained using computer-aided tomography (CT-scanning).

In some embodiments, the method further includes obtaining the measured anatomical data regarding the portion of the surface of the cranium. In some embodiments, manufacturing the implant from the radiolucent material based on the computer-aided design representation includes machining the cranial implant from a material using a multi-axis computer numerical control machine tool.

In some embodiments, a radiolucent implant may be provided with a standard contact surface, which is later machined, molded or formed to be complementary in shape to a measured portion of a cranium.

FIG. 33 schematically depicts another method 420 of making a cranial implant. For illustrative purposes, method 420 is described with respect to the reference numbers for head post 300 of FIG. 27; however, method 420 may be employed to make other types of head posts (e.g., head post 350) or other types of cranial implants (e.g., access chamber 10, stimulation chamber 200). A cranial implant having a standard contact surface is obtained (step 422). The cranial implant may be a radiolucent implant. The radiolucent implant may include a carbon-PEEK material. The standard contact surface may be planar, may have standard (non-customized) curvature, or may have any other initial shape. A CAD representation is created of a customized contact surface for the implant having a shape complementary to measured anatomical data regarding a portion of a surface of a particular cranium (step 424). As explained above, the measured anatomical data may be MRI data, X-ray data, CT scan data, etc. The standard contact surface of the implant is modified based on the CAD representation of the customized contact surface (step 426). For example, the CAD representation may be used in directions to control a multi-axis computer numerical control machine tool.

In some embodiments, the measured anatomical data regarding a portion of the surface of the cranium may be smoothed prior to creation of the CAD representation of the of the implant or of the customized contact surface of the implant. The smoothing kernel size may be chosen by the user based on the resolution of the measured anatomical data and/or based on the tolerances of the method used to produce the implant or to make the contact surface.

Cranial implants described herein may be formed, machined or modified using any suitable technique or combination of techniques. For example, an implant may formed entirely out of a single piece of material using a multi-axis CNC machine (e.g., a 3-axis or a 5-axis CNC machine). An implant may be molded or cast. An implant may be molded or cast and then machined. An implant may be formed using three-dimensional printing techniques. An implant may be produced using a combination of any of the aforementioned techniques. An implant may include multiple different pieces, any of which may be formed using one or more of the aforementioned techniques, which are then joined together or affixed to each other.

In some embodiments, an implant may be further processed to improve surface or bulk material properties after being formed. For example, an implant may be annealed after forming, which may alter a crystal structure of the material, remove any thermal history, limit subsequent dimensional changes at high temperature, and/or remove internal stresses. Such a thermal annealing is particularly useful for improving material properties in a carbon-PEEK implant after forming.

### EXAMPLES

### A. Method of Making Cranial Access Chambers

Cranial implants (specifically cranial recording chambers, cranial stimulation chambers and head posts) were made for craniums of multiple small primates. Initially, curvature of a cranium was derived using structural magnetic resonance imaging (MRI) for a specific primate. A software analysis package Analyze from AnalyzeDirect, Inc. was used to segment the surface of the cranium from the raw structural MRI data to create a stereolithography computer-aided design (STL-CAD) representation of the cranium of the specific primate. 3D CAD software programs, specifically SolidWorks from Dassault Systèmes SolidWorks Corp. of Waltham, Mass. and PowerSHAPE from Delcam Plc. of Birmingham, UK, were used to customize the configuration of the chamber to have a base with a contact surface that is complementary in shape to the measured anatomical data regarding a portion of a surface of the cranium of the specific primate based on the STL-CAD representation of the cranium of the specific primate.

For cranial access chambers, the location for each chamber is selected based on pre-determined anatomical targets in the brain. Within the model data, target vectors were placed on the surface of the cranium for each chamber at the exact position and orientation (stereotactic coordinates) above the desired brain tissue to be accessed. The configuration of the surface of the base was specific to the selected stereotaxic coordinates of the portion of the cranium to be fitted, and to the orientation of the chamber relative to the portion of the surface of the cranium, which were determined based on scientific or clinical goals (e.g., access to the lateral intraparietal area (LIP) of the parietal cortex, access to area V4 of the extrastriate visual cortex).

The customized design for the chamber was then converted to computer-aided machine (CAM) format, which was used by a multi-axis computer numerical control (CNC) machine tool to machine the chamber base out of a carbon-PEEK material, specifically, a 30% filled carbon-PEEK material provided by INVIBIO Ltd. of Lancashire, UK. The contact surface of the chamber base complemented the underlying cranium surface. A sterile bone void filler compound, specifically a bioactive ceramic putty containing salts of calcium, sodium, silica, and phosphorus (e.g., CONSIL Synthetic Bone Graft Putty from NUTRAMAX Laboratories, Inc. of Edgwood, Md.), was used to plug any small gaps beneath the base of the chamber. The largest gap between the cranial surface and the implant contact surface observed with any of the implants was less than 400 μm. Typically, the largest gap for an implant was less than 200 μm (e.g., approximately 50 to 200 μm).

### B. Recording Chamber with Microdrive

Recording chambers were manufactured in accordance with configurations depicted in FIGS. 1-5 using the method described above in part A. Although FIGS. 1-3 do not depict contact surfaces complementary in shape to measured anatomical data, the manufactured chambers had customized contact surfaces on bases, such as contact surface 44 on base 34 in FIG. 4 and contact surface 64 on base 54 of FIG. 5. In a manufactured recording chamber the outer diameter D₁ of the base was 31 mm and the inner diameter D₂ of the access channel 20 was 19 mm. The height h₁ of the upper portion 12 of the chamber was 10.16 mm. The height of the base 14 of the chamber h₂ was about 10 mm to about 25 mm varying depending on the particular curvature of the contact surface of the base and the orientation of the chamber relative to the portion of the cranium. The recording chamber had eight apertures 11a . . . 1h, which were countersunk screw guide holes for receiving bone screws.

For each recording chamber, a microdrive body 80 was machined out of carbon-PEEK. A proximal end surface 85 of each microdrive body was configured to extend past the contact surface of the corresponding base 14 of the recording chamber 10 by about 2 mm as illustrated by h₆ in FIG. 19. Although FIGS. 15 and 17 depict the microdrive body 80 with a proximal portion 88 having a flat surface 85 normal to an axis 41 of the access channel, some of the manufactured microdrives (e.g., the LIP chamber) had an angled surface 85 that was not normal to an axis 41 of the access channel as depicted in FIG. 19. Each microdrive body 80 included an array of guide holes 20, each guide hole was 350 μm in diameter and the guide holes were spaced 500 μm apart. The microdrive body 80 included a distal portion 86 with a height h₃ of 27.94 mm and a proximal portion 88 with a height h₄ of about 14 mm, which varied depending on the particular curvature of the contact surface of the base and the angle of orientation of the microdrive body with respect to the portion of the cranium. Other components of the microdrive were assembled as described above with respect to FIGS. 19-22-18.

Skull screws of the configuration depicted in FIGS. 9-11 were machined from carbon-PEEK and coated with hydroxyapatite for securing radiolucent implants (e.g., cranial access chambers and head posts) to a cranium. Each radiolucent skull screw was self-tapping (i.e., creates its own thread as it is advanced into bone) and therefore did not require that a surgeon tap the drilled screw hole prior to insertion. The thread diameter was 2.4 mm, the thread pitch was 1.0 mm, the core diameter was 1.7 mm, and the diameter of the head was 4.0 mm. The results described herein were obtained using uncoated titanium self-tapping screws to secure the implants to the cranium. However, Applicants plan to use the hydroxyapatite coated carbon-PEEK self-tapping screws that were manufactured as described above in future studies.

After a craniotomy was performed, guide holes were made in the cranium using a hand drill with a 1.8 mm bit diameter. Each recording chamber was affixed to the corresponding portion of a cranium using skull screws. Typically, a contact surface of the base of the recording chamber closely corresponded to the portion of the surface of the cranium, with gaps between the cranium surface and the recording chamber contact surface of typically about 100 μm and rarely exceeding 500 μm. The gaps were filled with the sterile bone void filler compound.

After the recording chamber was affixed to the cranium, the proximal portion 88 of the microdrive 80 was inserted into the recording chamber access channel 20 to form a recording assembly 100. The recording assembly also included a lid 104 covering a distal end 81 of the microdrive body.

### C. Stimulation Chamber

Stimulation chambers with configurations depicted in FIGS. 23-24 were manufactured according to a method described in section A above. Although contact surface 24 in FIGS. 23-24 is not complementary in shape to measured anatomical data as depicted, the manufactured chambers had customized contact surfaces, which corresponded to a measured shape of a portion of a surface of a cranium. The method for customizing the contact surface of the stimulation chamber is described above in part A. The stimulation chamber 210 had a rectangular perimeter of about 16.5 mm×15 mm with rounded corners. The first access channel 220 and the second access channel 221 of the stimulation chamber each had a 3.6 mm square opening with rounded corners. The height h₈ of the stimulation chamber was roughly 20 mm, but varied significantly at different points along its perimeter depending upon the particular curvature of the underlying cranium and the orientation of the chamber on the underlying cranium. The stimulation chamber included a first access channel 220 and the second access channel 221, each configured to receive an electrode guide.

After a craniotomy was performed, guide holes were made in the cranium using a hand drill with a 1.8 mm bit diameter, and the stimulation chamber 210 was affixed to the portion of the surface of the cranium using radiolucent skull screws. A first electrode guide 240 and a second electrode guide 241 were positioned in the first access channel 220 and the second access channel 221 respectively. The first electrode guide 240 and a second electrode guide 241 each had four guide channels to align stimulating electrodes with underlying brain structures forming a stimulation assembly 200. The stereotaxic coordinates and orientation of the stimulation chamber was selected for bilateral stimulation of reward-related structures of the brain (e.g., nucleus accumbens, VTA) near the midline. The stimulation assembly 200 further included a lid 250 that could be removed during use.

Typically, a contact surface of the base of the stimulation chamber closely corresponded to the portion of the surface of the cranium, with gaps between the cranium surface and the recording chamber contact surface of typically about 100 μm and rarely exceeding 500 μm. The gaps were filled with the sterile bone void filler compound.

### D. Head Post

During the initial experiments with animals, legged head posts with initially planer contact surfaces were made out of titanium. The legs were subsequently bent to roughly conform to the shape of the underlying cranium. Each head post was affixed to a cranium using titanium head screws.

### E. Results

Recording chambers and stimulation chambers were implanted on multiple animal subjects. Generally, the implanted recording chambers and stimulation chambers did not show recession of skin from the perimeter of the chamber even over extended periods of time. For example, in one animal subject, 1.7 years after implantation there was no observable skin recession and the skin adhered to the perimeter of the chamber at the skin implant boundary. Further, there was no sign of infection at the skin-implant boundary and there was only minimal granulated tissue (about 0.5 mm radially) at the wound margin, which may also be referred to as the skin-implant interface.

Conventional titanium head posts having radially extending legs bent to roughly conform to the underlying cranium were implanted on multiple animals. In some of the animals, the skin receded radially away from the center of the head post by 5-10 mm over the course of a year. A smaller number of animals experienced extreme recession of 28-32 mm radially exposing all 4 legs and underlying bone.

### F. Modified Prototype Examples

The inventors modified the configurations used in the initial experiment and produced prototype cranial access chambers and head posts based on the modified configurations. Specifically, access chambers based on the configuration depicted in FIGS. 6-8 were produced. For the revised configurations, a drill bit sleeve was used for drilling the holes in the cranium for the implant screws. The drill bit guide and the length of the apertures (e.g., the guide holes or screw holes) of the cranial access chamber lower portion provided sufficient stability for straight and stable drilling of screw holes (and insertion of screws) by providing a tightly constrained linear insertion path. The surgical drill set employed included cylindrical collars, referred to as drill bit sleeves, that prevented slippage of the drill bit on the bone and ensured a precisely-sized screw hold, which increased the purchase of the self-tapping screws. Using the long apertures of the chambers and the drill bit sleeves, the inventors were able to drill screw holes into the bone at oblique angles of approach (e.g., at 45 degrees) without slippage or travel of the drill bit. The drill bit sleeves employed had an outer diameter of 2.39 mm, which was slightly smaller than the aperture diameter of 2.5 mm. The drill bit sleeve had an inner diameter of 1.73 mm, which was slightly larger than the diameter of the drill bit used for the revised implant configurations. In the revised configurations, each screw had a screw length of 10 mm. To achieve the desired extension of the screw beyond the contact surface of the implant into the bone, the counter-sink depth of each aperture was adjusted during the CAD design process. In other embodiments, screws of different lengths may be employed and the apertures may not be counter-sunk.

A prototype carbon-PEEK legged head post was produced according to the configuration of FIGS. 25-28. The head post had a contact surface 318 with a shape complementary to a measured portion of a surface of the cranium using the process described above. Unlike the access chambers, which were produced using a 3-axis CNC machine tool, the revised configuration had apertures with an insertion axis of each aperture being perpendicular to the surface of the cranium requiring the use of a 5-axis CNC machine tool. The two additional rotational axes of the 5-axis CNC machine enabled each aperture to be exactly perpendicular to the cranium at its point of entry (see detail of FIG. 28). Driving each screw into the cranium with a straight, surface normal approach increases the purchase of the screw into the bone and reduces shear forces on the implanted screw, strengthening the attachment of the implant.

Another prototype carbon-PEEK continuous custom shaped head post was produced according to the configuration of FIGS. 29 and 30. This continuous head post configuration had a smaller lateral extent and a corresponding smaller effective footprint on the cranium than the legged head post configuration leaving more remaining cranium available for additional chamber access. Unlike the legged head post configuration in which the skin extends over the surface of the legs, the base of the continuous head post may be raised (e.g., to 1 cm above the cranium) to provide a well-defined implant boundary for the skin margin to abut. Finally, the continuous surface configuration benefits from the ability to have additional screw holes to more robustly secure the head post to the cranium.

FIGS. 34-36 illustrate models and manufactured examples of the revised configurations. FIG. 34 depicts a computer model of measured anatomical data 450, a custom shaped head post 460, a first access chamber 470, and a second access chamber 480. FIG. 35 depicts the smoothed anatomical data 452 used to produce the model for corresponding contact surfaces of the implants. In FIG. 35, first access chamber 470 and second access chamber 480 are translucent to depict an axis 472 of the first access chamber and an axis 482 of the second access chamber, as well as locations of the apertures. FIG. 36 is an image of the carbon-PEEK head post 460, the carbon-PEEK first access chamber 470 and the carbon-PEEK second access chamber 480 produced using the model depicted in FIG. 35.

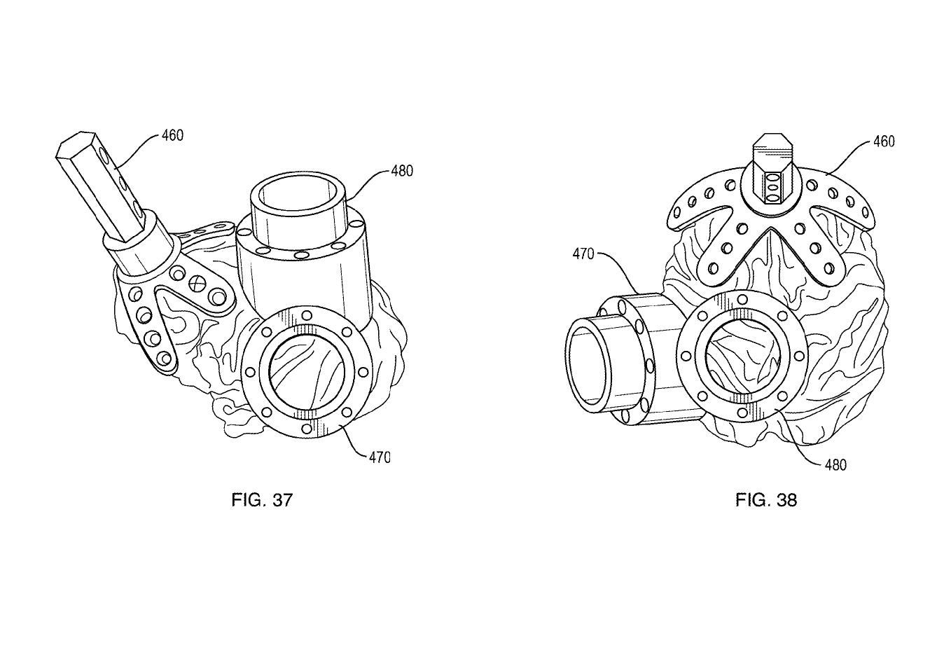

FIGS. 37 and 38 depict the different portions of the brain targeted by the first access chamber 470 and the second access chamber 480. FIG. 37 depicts the implants and the brain as view along axis 472 of the first access chamber 470. As depicted in FIG. 37, the first access chamber 470 is configured and positioned for targeting the lower-right quadrant of visual field representation in V4 with the center of the access channel between the lunate and superior temporal sulcus. FIG. 38 depicts the implants and the brain as viewed along axis 482 of the second access chamber 480. As depicted in FIG. 38, the second access chamber 480 is configured and positioned for targeting the intraparietal sulcus and area 5 of the brain and would be suitable for a dorsal approach to the lateral intraparietal area.

Although some examples described above are directed to radiolucent implants, the disclosure herein also applies to cranial implants that do not include radiolucent materials. Disclosure herein regarding a cranial implant (e.g., a cranial access chamber or a head post) having a contact surface with a shape complementary to a measured surface of a portion of a cranium, and to methods of producing such an implant, applies both to radiolucent implants and to non-radiolucent implants (e.g., titanium implants). Further, disclosure herein regarding a chronic cranial implant (e.g., a cranial access chamber or a head post) that has an outer perimeter shaped to form a gapless interface with surrounding tissue (e.g., an outer perimeter that is a convex shape or is approximately circular, oval or elliptical in shape) applies both to radiolucent implants and to non-radiolucent implants (e.g., titanium implants).

Embodiments may be employed in research and/or clinical applications. For example, the growing fields of neural prosthetics and brain-machine interfaces will benefit from robust, MRI-compatible implants for chronically accessing brain tissue to treat neurological disorders. For example, the relatively well-established treatment for Parkinson's disease, deep-brain stimulation (DBS), has paved a path for potentially treating many other neurological disorders using electrophysiological brain recording and stimulation. Cranial implants made from carbon-PEEK may offer many advantages over existing titanium (and other ‘softer’ plastic) cranial implants for applications involving MRI or X-ray imaging. Further, cranial implants described herein incorporate configurations that promote a smooth uninterrupted implant/skin interface and reduce the likelihood of skin recession increasing the lifetime of the implant and reducing the risk of infection and bone degradation for stable and reliable long-term recording and stimulation of neural structures.

Cranial implants disclosed herein may be employed for research, diagnostic testing, and/or treatment of non-human and human subjects. Any reference to a cranium or a skull herein may be interpreted as a reference to a cranium or a skull of a human subject or of a non-human subject. A subject may be a primate, or a non-primate vertebrate.

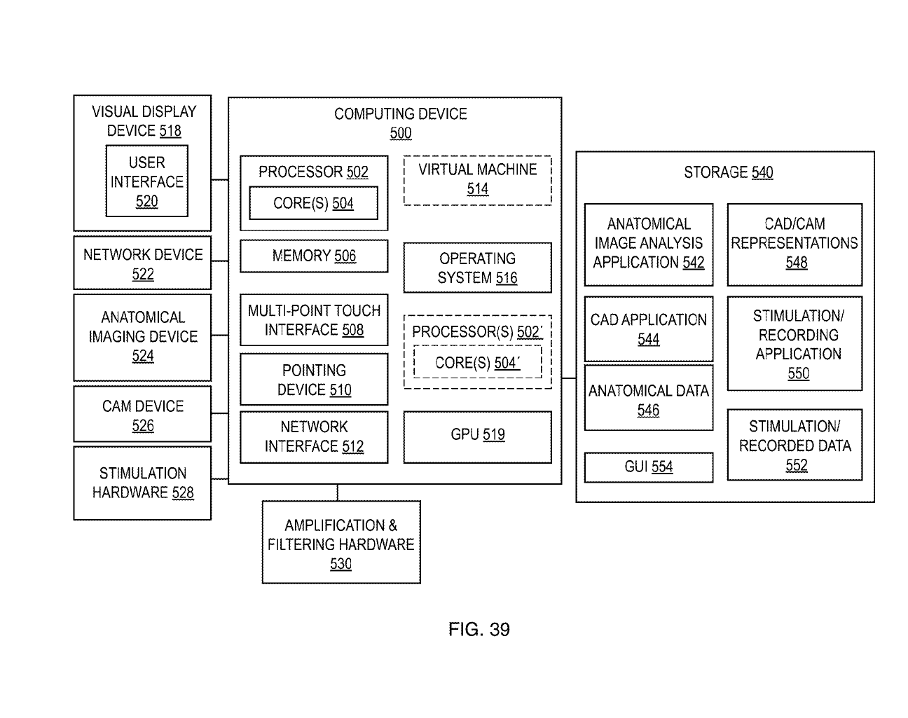

FIG. 39 is a block diagram of an exemplary computing device 500 that may be employed when performing any of the methods provided by exemplary embodiments. The computing device 500 may be any computer system, such as a workstation, desktop computer, server, laptop, handheld computer, tablet computer (e.g., the iPad™ tablet computer), mobile computing or communication device (e.g., the iPhone™ mobile communication device, the Android™ mobile communication device, and the like), or other form of computing or telecommunications device that is capable of communication and that has sufficient processor power and memory capacity to perform the operations described herein.

The computing device 500 includes one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, one or more optical disks, one or more USB flash drives), and the like. For example, memory 506 included in the computing device 500 may store computer-readable and computer-executable instructions or software for implementing exemplary embodiments. The computing device 500 also includes processor 502 and associated core 504, and optionally, one or more additional processor(s) 502′ and associated core(s) 504′ (for example, in the case of computer systems having multiple processors/cores), for executing computer-readable and computer-executable instructions or software stored in the memory 506 and other programs for controlling system hardware. Processor 502 and processor(s) 502′ may each be a single core processor or multiple core (504 and 504′) processor. Computing device 500 may optionally include a graphical processing unit (GPU) 519 for analyzing and displaying image information.

Virtualization may be employed in the computing device 500 so that infrastructure and resources in the computing device may be shared dynamically. A virtual machine 514 may be provided to handle a process running on multiple processors so that the process appears to be using only one computing resource rather than multiple computing resources. Multiple virtual machines may also be used with one processor.

Memory 506 may include a computer system memory or random access memory, such as DRAM, SRAM, EDO RAM, and the like. Memory 506 may include other types of memory as well, or combinations thereof.

A user may interact with the computing device 500 through a visual display device 518, such as a screen or monitor, that may display one or more user interfaces 520 that may be provided in accordance with exemplary embodiments. The visual display device 518 may also display other aspects, elements and/or information or data associated with exemplary embodiments, for example, images of anatomical data, CAD representations of anatomical data, CAD representations of implants. The visual display device 518 may be a touch-sensitive display. The computing device 500 may include other I/O devices for receiving input from a user, for example, a keyboard or any suitable multi-point touch interface 508, a pointing device 510 (e.g., a mouse, a user's finger interfacing directly with a display device, etc.). The keyboard 508 and the pointing device 510 may be coupled to the visual display device 518. The computing device 500 may include other suitable conventional I/O peripherals.

In some embodiments, the computing device 500 may be connected with one or more anatomical imaging device(s) 524 that provide anatomical data (e.g., MRI data, CT scan data). In some embodiments, the computing device 500 may be connected with a CAM Device 526 that is used in Computer-Aided Machining. In some embodiments, the computing device 500 may be connected with Stimulation Hardware 528 for generating electrical or optical stimulation and/or Amplification & Filtering Hardware 530 for amplifying or filtering electrical signal from electrodes.

The computing device 500 may include one or more storage devices 540, such as a hard-drive, CD-ROM, or other computer readable media, for storing data and computer-readable instructions and/or software that implement exemplary embodiments as taught herein. The storage device 540 may be provided on the computing device 500 or provided separately or remotely from the computing device 500.

Exemplary storage device 540 may store any suitable information required to implement exemplary embodiments. For example, the storage 540 may store computer-readable computer-executable instructions for implementing applications to be executed by the computing device (described as “applications” herein) and data generated by or obtained from the applications, as well as data supplied from other sources. For example, storage 540 may store an Anatomical Image Analysis Application 542, input anatomical data and processed anatomical data produced by the application (collectively Anatomical Data 546). As another example, storage 526 may include a CAD Application 544 that creates CAD representations from anatomical data and/or that creates CAD representations of implants from CAD representations of anatomical data. The storage 540 may store CAD representations and/or CAM representations 548. In some embodiments, the storage 540 may Stimulation/Recording Application 550 for stimulation using the implant or recording signals from electrodes associated with the implant. In some embodiments, the storage 540 may store instructions for stimulation or stimulation data and/or recorded data 552. In some embodiments, storage 540 may also store computer-readable computer-executable instructions for implementing a graphical user interface 554.

The computing device 500 may include a network interface 512 configured to interface via one or more network devices 522 with one or more networks, for example, Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections, controller area network (CAN), or some combination of any or all of the above. The network interface 512 may include a built-in network adapter, network interface card, PCMCIA network card, card bus network adapter, wireless network adapter, USB network adapter, modem or any other device suitable for interfacing the computing device 500 to any type of network capable of communication and performing the operations described herein. The network device 522 may include one or more suitable devices for receiving and transmitting communications over the network including, but not limited to, one or more receivers, one or more transmitters, one or more transceivers, one or more antennae, and the like. Anatomical data, CAD representation data, CAM data, stimulation data for the implant, and/or recorded data from implant electrodes may be obtained via the network device 522.

The computing device 500 may run any operating system 516, such as any of the versions of the Microsoft® Windows® operating systems, the different releases of the Unix and Linux operating systems, any version of the MacOS® for Macintosh computers, any embedded operating system, any real-time operating system, any open source operating system, any proprietary operating system, any operating systems for mobile computing devices, or any other operating system capable of running on the computing device and performing the operations described herein. In exemplary embodiments, the operating system 516 may be run in native mode or emulated mode. In an exemplary embodiment, the operating system 516 may be run on one or more cloud machine instances.

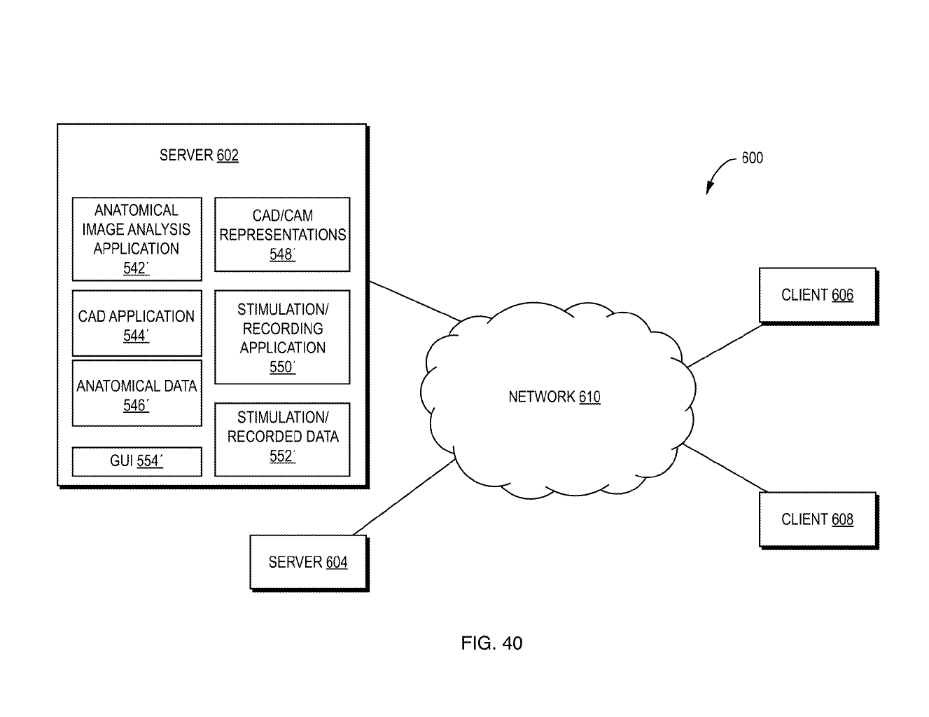

FIG. 40 is a diagram of an exemplary network environment 600 suitable for a distributed implementation of exemplary embodiments. The network environment 600 may include one or more servers 602 and 604 coupled to one or more clients 606 and 608 via a communication network 610. The network interface 512 and the network device 522 of the computing device 500 enable the servers 602 and 604 to communicate with the clients 606 and 608 via the communication network 610. The communication network 610 may include, but is not limited to, the Internet, an intranet, a LAN (Local Area Network), a WAN (Wide Area Network), a MAN (Metropolitan Area Network), a wireless network, an optical network, and the like.

The communication facilities provided by the communication network 610 are capable of supporting distributed implementations of exemplary embodiments. Applications and interfaces may be provided to a client by a server via the network. For example, server 602 and/or server 604 may provide any or all of an anatomical image analysis application 542′, a CAD application 544′, a stimulation/recording application 550′, and a GUI 554′. Storage of and access to data may be provided to a client or to another server by a server via the network. For example, server 602 and/or server 604 may provide any or all of anatomical data 546′, CAD/CAM representations 548′, and stimulation/recorded data 552′.

In an exemplary embodiment, the servers 602 and 604 may provide the clients 606 and 608 with computer-readable and/or computer-executable components, products, or services under a particular condition, such as a license agreement. The computer-readable and/or computer-executable components or products may include those for providing and rendering an exemplary graphical user interface for video surveillance of video data obtained locally or accessed via a network. The clients 606 and 608 may provide and render an exemplary graphical user interface using the computer-readable and/or computer-executable components, products and/or services provided by the servers 602 and 604. In an exemplary embodiment, the clients 606 and 608 may transmit measured anatomical information regarding a cranium to the servers 602 and 604 that may, in turn, generate a CAD representation of the cranium and/or generate a CAD representation of a contact surface of an implant corresponding to the representation of the cranium.

Alternatively, in another exemplary embodiment, the clients 606 and 608 may provide the servers 602 and 604 with computer-readable and computer-executable components, products and/or services under a particular condition, such as a license agreement. The servers 602 and 604 may provide and render an exemplary graphical user interface using the computer-readable and/or computer-executable components, products and/or services by the clients 606 and 608. In an exemplary embodiment, the servers 602 and 604 may transmit information regarding a CAD representation of at least a contact surface of an implant or regarding a CAM representation of at least a contact surface of an implant.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements, device components or method steps, those elements, components or steps may be replaced with a single element, component or step. Likewise, a single element, component or step may be replaced with a plurality of elements, components or steps that serve the same purpose. Moreover, while exemplary embodiments have been depicted and described with references to particular embodiments thereof, those of ordinary skill in the art will understand that various substitutions and alterations in form and detail may be made therein without departing from the scope of the invention. Further still, other aspects, functions and advantages are also within the scope of the invention. Exemplary flowcharts are provided herein for illustrative purposes and are non-limiting examples of methods. One of ordinary skill in the art will recognize that exemplary methods may include more or fewer steps than those illustrated in the exemplary flowcharts, and that the steps in the exemplary flowcharts may be performed in a different order than the order depicted in the illustrative flowcharts.

Those skilled in the art in view of the present disclosure will readily appreciate that various elements of the embodiments described herein may be modified and combined in multiple different ways without materially departing from this disclosure. Accordingly, all such modifications and combinations are intended to be included within the scope of this disclosure.

1.-22. (canceled)

23. A cranial access chamber, the chamber comprising:
an upper portion; and
a base portion comprising:
a sidewall defining a plurality of apertures extending through the base portion, each aperture configured to receive a fixation element, an inner surface of the sidewall and the upper portion defining an access channel; and
a contact surface complementary in shape to a measured surface of a portion of a cranium.

24. The cranial access chamber of claim 23, wherein a shape of the contact surface is formed based on the measured anatomical data of the surface of the portion of the cranium.

25. The cranial access chamber of claim 23, wherein the contact surface is shaped such that the largest gap formed between the contact surface and measured surface of the portion of the cranium would be less than 500 μm.

26. The cranial access chamber of claim 25, wherein the contact surface is shaped such that largest gap formed between the contact surface and measured surface of the portion of the cranium would be less than 400 μm.

27. The cranial access chamber of claim 26, wherein the contact surface is shaped such that largest gap formed between the contact surface and measured surface of the portion of the cranium would be less than 200 μm.

28. The cranial access chamber of claim 23, wherein the contact surface comprises an osteoconductive coating.

29. The cranial access chamber of claim 23, wherein the base portion has an outer perimeter shaped to form a gapless interface with surrounding tissue.

30. (canceled)

31. The cranial access chamber of claim 29, wherein the outer perimeter forms a convex shape.

32. The cranial access chamber of claim 23, wherein the cranial access chamber is configured for chronic attachment to a portion of a cranium.

33. A head post comprising:
an upper portion; and
a base portion defining a plurality of apertures extending through the base portion, each aperture configured to receive a fixation element, the base portion having a contact surface complementary in shape to a measured surface of a portion of a cranium.

34. The head post of claim 33, wherein in at least the base portion comprises a radiolucent material.

35. The head post of claim 34, wherein the radiolucent material is carbon-reinforced PolyEtherEtherKetone (carbon-PEEK).

36. The head post of claim 33, wherein a shape of the contact surface is formed based on measured anatomical data of the surface of the portion of the cranium.

37. The head post of claim 33, wherein the contact surface is shaped such that the largest gap formed between the contact surface and measured surface of the portion of the cranium would be less than 500 μm.

38. The head post of claim 37, wherein the contact surface is shaped such that largest gap formed between the contact surface and measured surface of the portion of the cranium would be less than 400 μm.

39. The head post of claim 37, wherein the contact surface is shaped such that largest gap formed between the contact surface and measured surface of the portion of the cranium would be less than 200 μm.

40. The head post of claim 33, wherein an outer perimeter of the base portion forms a convex shape.

41. The head post of claim 40, wherein the base portion has an outer perimeter shaped to form a gapless interface with surrounding tissue.

42. (canceled)

43. The head post of claim 33, wherein the contact surface comprises an osteoconductive coating.

44. The head post of claim 33, wherein the head post is configured for chronic attachment to a portion of a cranium.

45.-71. (canceled)

72. The cranial access chamber of claim 23, wherein the upper portion and the base portion comprise a radiolucent material.

73. The cranial access chamber of claim 72, wherein the radiolucent material is carbon-reinforced PolyEtherEtherKetone (carbon-PEEK).

74. The cranial access chamber of claim 72, wherein the radiolucent material comprises a conductive radiolucent material.

75. The cranial access chamber of claim 23, wherein the cranial access chamber is configured to receive at least a portion of a multi-channel microdrive for guiding one or more electrodes.

76. The cranial access chamber of claim 23, wherein the cranial access chamber is a recording chamber.

77. The cranial access chamber of claim 23, wherein the chamber is configured for electrophysiological recordings.

78. The cranial access chamber claim 23, wherein the cranial access chamber is configured to receive one or more guide elements, each guide element defining one or more electrode guide holes.

79. The radiolucent cranial access chamber claim 23, wherein the cranial access chamber is a stimulation chamber.

80. The cranial access chamber of claim 23, further comprising a lid configured to cover the upper portion of the access channel.

81. The cranial access chamber of claim 23, wherein the chamber is configured for chronic attachment to a portion of a cranium without the use of an adhesive or bonding agent.
